SEQUENCE LISTING (7 sequences)

SEQ ID NO: 1 - DNA, 2532 bp, Saccharomyces cerevisiae
atgggtaactatgattcaactccaattgcaaagtcagatagaattaagagattagttgaccacttatatgctaaaatgccagaaatagaggcagcaagggctgaattaataacagaatcttttaaagcaacagagggacagcctgttgttatgaggaaagccagggccttcgaacatattttaaagaatttgcctattattattagaccagaagaattgatcgttggttcaactacaattgcaccaagaggatgtcaaacatatcctgaattttcatacgaatggttagaggccgaatttgaaactgttgagaccagatcagcagacccattctatatttcagaggaaacaaagaagagattattggcagcagatgcatattggaagggtaaaacaacatctgaattggctacctcatatatggcccctgaaaccttgagagctatgaagcataacttctttacaccaggaaactacttctataatggtgttggacatgttactgtccagtacgaaactgtcttggctatcggtttgaatggagtcaaggagaaagttagaaaagaaatggagaactgccattttggtgatgcagactattcaactaaaatgtgcttcttagagtctattttaatttcttgtgacgctgttataacctatgccaacagatacgctaaaatggccgaggaaatggctgaaaaggagacagatgcagctagaagacaggaattgttgacaattgcaagagtttgtaagaacgtccctgaatttcctgccgaatctttccaagaagcctgtcaatctttctggtttatacaacaagtcttgcaaatagagtcttctggtcattctatttctccaggtagatttgatcaatacatgtacccatattatgagaaagatttgaaggagggttcattgactagggaatatgcacaagagttgatagattgtatatgggttaagttgaacgacttaaacaaatgcagggacgccgcttctgctgaaggttttgcaggttattcattgttccagaatttgatagtcggtggtcaaacagtacagggtagagatgcaaccaatgacttgtctttcatgtgtatcactgcctcagaacacgtcttcttaccaatgccttcattatctattagagtttggcacggttcttcaaaggccttgttgatgagagccgcagaattgaccagaacaggaatcggattgccagcttattacaatgatgaagttattataccagctttggttcatagaggtgctacaatggacgaagcaagaaattacaatataataggatgtgttgagcctcaagttccaggtaaaacagatggatggcatgatgctgctttctttaatatgtgtagacctttagaaatggtcttttcaaacggttacgacaacggtgaaatagcctcaattcagactggtaatgttgaatcatttcagtcatttgatgagttcatggaagcctacaggaaacaaatgttatataatattgaattgatggtcaatgctgacaacgccattgactacgctcacgccaaattggcccctttgcctttcgagtcttgtttggtcgatgattgtattaagagaggaatgtcagcacaagaaggtggtgcaatatacaattttaccggtccacaaggattcggtattgctaatgtagctgattctttgtataccataaagaagttggtatttgaagaaaagagaattaccatgggtgagttgaagaaggccttggaaatgaactacggaaagggattggacgcaacaacagcaggagacattgctatgcaggtcgcaaagggtttgaaagacgccggtcaagaagtcggacctgatgtcatagctaatactattagacaagttttggaaatggaattgcctgaggatgtcaggaagagatatgaggaaattcatgagatgatattggaattaccaaaatacggtaatgatattgacgaagttgacgaattagctagagaagccgcatacttttataccaggccattagaaacattcaagaacccaaggggaggtatgtatcaagcaggtttgtacccagtctctgccaatgttcctttaggtgcacaaaccggtgcaacaccagacggtagattggcccatacacctgttgcagatggagtaggtccaacatcaggatttgatatttctggaccaactgcctcttgtaactctgtcgccaaattagatcatgctattgcatctaacggtaccttgtttaatatgaagatgcatcctactgctatggccggagaaaagggtttggaatctttcatctctttaattagaggttattttgatcaacagggaatgcacatgcaattcaatgttgttgacagggccactttattagatgctcaagctcaccctgagaagtactctggattgattgttagggtagcaggttattctgctttattcaccaccttatctaagtctttacaagatgatataatcaagaggacagaacaagctgacaatagataa

SEQ ID NO: 2 - DNA, 795 bp, Saccharomyces cerevisiae
atgaaggaatatttgaatacctctggaagaattttcgatatacagaggtactctattcatgatggacctggtgtaaggacaatagtattcttgaagggatgtgctttaaggtgcagatggtgctgcaatccagagtcacaatcatttgaagttgaaacaatgacaattaatggtaagcctaaggttatgggtaaagacgtcacagtagccgaagttatgaaaaccgttgaaagagatatgccatattacttacaatcaggtggtggtattaccttatcaggaggagaatgcacattacaaccagaattttcattgggattattgagggctgctaaagacttaggtatctctactgccatagaatcaatggcctacgctaagtatgaagtcatcgaaactttgttgccatacttggacacctacttaatggatattaaacacatgaacccagaaaagcataaggagtacactggacatgataacttaaggatgttggagaacgccttaagagtcgcacactctggtcaaactgaattaattattagggtccctgttatcccaggtttcaacgctacagaacaagaattattagatattgccaagttcgccgatactttaccaggtgtaaggcagatacacatcttgccttatcacaatttcggtcaaggaaaatacgaaggtttgaatagagattaccctatgggtgatactgaaaagccttctaatgaacagatgaaggcctttcaagaaatgatacaaaagaatacttcattgcattgccaaatcggtggttaa

SEQ ID NO: 3 - DNA, 2532 bp, Escherichia coli
atgggtaattatgattcaacgccaatagccaaaagcgatcgtattaaacgcttagttgaccacctgtacgccaagatgccggaaatcgaggccgcacgcgctgagctgatcacggagagttttaaagccaccgaggggcagcctgtcgttatgcgtaaggcacgtgcgtttgaacacattttaaagaacctgccaatcattatacgtcctgaagaactgattgttggaagcactacgatcgcccctcgcgggtgtcagacctatccggagtttagttatgaatggctggaagctgagtttgagactgttgaaactcgctctgcagatccattctatatttctgaggaaacgaagaaacgcctgctggccgcggatgcttattggaaaggcaaaactacttctgaactggctacctcttatatggcaccggaaacactgcgcgcaatgaagcataatttctttaccccaggtaattatttctataatggcgtaggccatgtgacggtgcagtatgaaacggtactggcaattggcctgaatggagtaaaagagaaggtacgtaaagagatggaaaactgtcattttggtgatgcagactacagcactaagatgtgtttcctggagtcaattctgattagttgtgatgctgttataacttatgcgaatcgttatgccaaaatggcagaagaaatggcagaaaaggaaaccgacgcagcccgtcgccaggaactgctgaccatagcgcgtgtatgtaagaacgtacctgaatttcctgcagaatcttttcaggaggcttgtcaatcattttggttcattcagcaggtcttacagatcgagagttcagggcattcaatcagcccaggccgctttgaccagtatatgtatccgtattatgagaaggacctgaaggaaggttcactgacccgcgaatatgcacaggaactgattgattgcatttgggttaaactgaatgatctgaataagtgccgcgacgcagcgtcagccgagggctttgcgggttatagcctgtttcaaaatctgatcgttggtggacagactgttcaaggtcgtgatgccactaacgacctgagctttatgtgtattactgcaagtgagcacgtgttcctgccgatgccgagcttatcaattcgcgtttggcacggctcaagcaaggctttactgatgcgcgccgctgagctgacacgcacaggtattggcctgccggcctattataatgatgaagtcattatacctgccctggttcatcgtggtgcaacaatggatgaggcgcgcaactataacattatcggttgtgtggaaccacaggtacctgggaaaaccgatggttggcacgacgcggcgttctttaatatgtgtcgtccgctggagatggttttcagtaatggctacgataatggggagattgcaagcattcagactggcaatgtggagtctttccagtcatttgacgagtttatggaagcctaccgtaaacaaatgctgtataatatagagctgatggttaatgcagataatgctatagattacgcacatgccaaattagccccgctgccgtttgaaagctgtctggtagatgattgcattaaacgcggtatgagcgcccaggaaggcggcgcgatatataatttcacgggtccacaggggtttggtatagcaaacgttgcagatagcctgtacacaataaagaaattagtgttcgaagaaaagcgcatcacaatgggagaactgaagaaagcactggaaatgaattatggaaaggggttagatgcaacaaccgcgggtgacattgcaatgcaggtcgccaaagggctgaaggacgcgggacaggaggtaggtccggacgtcatcgcgaataccattcgtcaggtgctggaaatggaactgccggaagatgttcgtaaacgctatgaagaaattcacgagatgattctggaattaccgaaatatggcaacgatatagatgaggtggatgaactggcccgcgaagcggcctacttttatacccgccctctggaaacgtttaagaatccgcgcggcggtatgtatcaggcgggtctgtacccggtgagtgccaatgtgccactgggagcacaaacgggtgcgacgcctgatggtcgcctggcgcacacgccagtagcggatggagttggtcctacttctgggtttgatatatctggtccgaccgcatcatgcaattcagttgcgaaactggatcatgccatcgcaagcaacggcaccctgttcaatatgaaaatgcaccctaccgcgatggcaggagaaaagggtctggagagtttcataagtctgattcgcggctattttgatcagcagggcatgcacatgcagttcaatgttgtcgatcgcgcaactctgctggatgctcaggcgcaccctgagaaatacagcggtctgattgttcgcgtggccggctattctgcactgttcaccactctgtctaaaagcctgcaggacgatataataaaacgtacggaacaagctgataaccgttaa

SEQ ID NO: 4 - DNA, 795 bp, Escherichia coli
atgaaggaatatctgaatacgagcggtcgcatcttcgatattcaacgttatagtatccatgatgggccaggcgtccgcacaatcgtctttctgaaaggctgcgcactgcgttgtcgctggtgttgtaatcctgaaagccaaagttttgaggttgaaaccatgactattaacggaaaaccaaaagtgatgggtaaagatgtaaccgttgccgaagttatgaaaacggttgaacgtgatatgccatattatctgcaatctggaggtggcatcacattatcaggtggtgaatgcacattacagccggagttttcattaggactgctgcgtgcagcaaaagacttaggcatttcaaccgctatcgaatcaatggcgtatgccaagtacgaagtaatagaaacactgctgccttacctggatacgtatctgatggacatcaagcacatgaaccctgagaaacataaagaatataccggccatgacaatctgcgtatgctggagaacgctctgcgcgtggcgcatagtggtcagacggaattaattattcgcgttcctgtgattccgggttttaacgcgacggaacaggaactgttagacattgctaagtttgcggatacgttaccgggtgtccgccagatccatatcctgccataccataatttcggacaaggcaaatacgaaggcctgaaccgtgattaccctatgggagacacggaaaagccgagcaacgaacagatgaaggcattccaggaaatgattcaaaagaacaccagtctgcattgtcaaataggaggttaa

SEQ ID NO: 5 - protein, 843 aa, Roseburia inulinivorans
MGNYDSTPIAKSDRIKRLVDHLYAKMPEIEAARAELITESFKATEGQPVVMRKARAFEHILKNLPIIIRPEELIVGSTTIAPRGCQTYPEFSYEWLEAEFETVETRSADPFYISEETKKRLLAADAYWKGKTTSELATSYMAPETLRAMKHNFFTPGNYFYNGVGHVTVQYETVLAIGLNGVKEKVRKEMENCHFGDADYSTKMCFLESILISCDAVITYANRYAKMAEEMAEKETDAARRQELLTIARVCKNVPEFPAESFQEACQSFWFIQQVLQIESSGHSISPGRFDQYMYPYYEKDLKEGSLTREYAQELIDCIWVKLNDLNKCRDAASAEGFAGYSLFQNLIVGGQTVQGRDATNDLSFMCITASEHVFLPMPSLSIRVWHGSSKALLMRAAELTRTGIGLPAYYNDEVIIPALVHRGATMDEARNYNIIGCVEPQVPGKTDGWHDAAFFNMCRPLEMVFSNGYDNGEIASIQTGNVESFQSFDEFMEAYRKQMLYNIELMVNADNAIDYAHAKLAPLPFESCLVDDCIKRGMSAQEGGAIYNFTGPQGFGIANVADSLYTIKKLVFEEKRITMGELKKALEMNYGKGLDATTAGDIAMQVAKGLKDAGQEVGPDVIANTIRQVLEMELPEDVRKRYEEIHEMILELPKYGNDIDEVDELAREAAYFYTRPLETFKNPRGGMYQAGLYPVSANVPLGAQTGATPDGRLAHTPVADGVGPTSGFDISGPTASCNSVAKLDHAIASNGTLFNMKMHPTAMAGEKGLESFISLIRGYFDQQGMHMQFNVVDRATLLDAQAHPEKYSGLIVRVAGYSALFTTLSKSLQDDIIKRTEQADNR

SEQ ID NO: 6 - protein, 264 aa, Roseburia inulinivorans
MKEYLNTSGRIFDIQRYSIHDGPGVRTIVFLKGCALRCRWCCNPESQSFEVETMTINGKPKVMGKDVTVAEVMKTVERDMPYYLQSGGGITLSGGECTLQPEFSLGLLRAAKDLGISTAIESMAYAKYEVIETLLPYLDTYLMDIKHMNPEKHKEYTGHDNLRMLENALRVAHSGQTELIIRVPVIPGFNATEQELLDIAKFADTLPGVRQIHILPYHNFGQGKYEGLNRDYPMGDTEKPSNEQMKAFQEMIQKNTSLHCQIGG

SEQ ID NO: 7 - DNA, 10 bp, Escherichia coli
mshhhhhgsg

(19) United States
(12) Patent Application Publication
(10) Pub. No.: US 2016/0040196 A1
(43) Pub. Date: Feb. 11, 2016

(54) ENZYMATIC CONVERSION OF BOTH ENANTIOMERS OF 1,2-PROPANEDIOL TO PROPIONALDEHYDE

(71) Applicant: University of Georgia Research Foundation, Inc., Athens, GA (US)

(72) Inventor: William Lanzilotta, Athens, GA (US)

(21) Appl. No.: 14/823,030

(22) Filed: Aug. 11, 2015

Related U.S. Application Data
(60) Provisional application No. 62/036,324, filed on Aug. 12, 2014.

(51) Int. Cl. C12P 7/24; C12N 9/88; C12N 9/04

(52) U.S. Cl. CPC C12P 7/24; C12N 9/0006; C12N 9/88; C12Y 101/01001; C12Y 402/01028

(57) ABSTRACT

Engineered organisms, cell-free enzyme mixtures, and methods are provided for converting both enantiomers of 1,2-propanediol to propionaldehyde. Engineered organisms are provided that convert both enantiomers of 1,2-propanediol to propionaldehyde but do not convert glycerol to 3-hydroxypropionaldehyde and/or do not convert propanal to propanol. The engineered organisms and cell-free enzyme mixtures can contain a diol dehydratase enzyme similar in sequence identity to Roseburia inulinivorans diol dehydratase. The engineered organisms and cell-free enzyme mixtures can contain a diol dehydratase activating enzyme similar in sequence identity to Roseburia inulinivorans diol dehydratase activating enzyme. Methods of converting both enantiomers of 1-2-propanediol to propanol can include culturing a microorganism provided herein under conditions and for a period of time sufficient to convert the 1,2-propanediol to propanol. The conditions can include a substantially anaerobic culture medium.

### CROSS-REFERENCE TO RELATED APPLICATIONS

This application claims priority to U.S. Provisional Patent Application U.S. Ser. No. 62/036,324 entitled “ENZYMATIC CONVERSION OF BOTH ENANTIOMERS OF 1,2-PROPANEDIOL TO PROPIONALDEHYDE” filed on Aug. 12, 2014 which is hereby incorporated by reference in its entirety.

### SEQUENCE LISTING

This application contains a sequence listing filed in electronic form as an ASCII.txt file entitled 02254612.txt, created on Aug. 11, 2015, and having a size of 19,271 bytes. The content of the sequence listing is incorporated herein in its entirety.

### BACKGROUND

Propanol is a chemical monomer that has significant value as a chemical intermediate and as a solvent for waxes, polyamides, natural and synthetic resins, polyacrylonitrile, cellulose esters and in the production of propionic acid, propionic aldehyde, propyl acetate, and propylamine. Propanol has utility in biofuel production and is also used in manufacturing pesticides and surface-active substances. Current enzyme-based or “green” technology available for the conversion of 1,2-propanediol into propionaldehyde, using enzymes in engineered organisms or enzymes in cell-free reactors has at least three pitfalls. First, all metabolic pathways and engineered organisms utilizing genes coding for B₁₂-dependent enzymes must either use cellular energy to synthesize the B₁₂ cofactor or that must add this expensive cofactor to the fermentation vessel or cell-free reactor. Second, the B₁₂-independent glycerol- and diol-dehydratase by Soucaille et al. would be detrimental to propanal or propanol production because glycerol is a substrate and a central metabolite during anaerobic metabolism (WO 2001004324 A1 20010118; WO 2008052595 A1 2008052595). In addition, the B₁₂-independent dehydratase by Soucaille et al. is used in the conversion of glycerol to 3-hydroxypropionaldehyde for 1,3-propanediol production and only uses S-1,2-propanediol as a substrate. Only one other B₁₂-independent dehydratase has been described for commodity chemical production. However, the enzyme identified by LANZATECH converts each enantiomer of 1,2-propanediol to a different product (International Publication Number WO2014/036152). Specifically, their enzyme converts S-1,2-propanediol and R-1,2-propanediol to propionaldehyde(propanal) and acetone respectively. There remains a need for improved methods and systems that can generate propionaldehyde from both enantiomers of 1,2-propanediol.

It is therefore an object of this disclosure to provide systems and methods for converting both enantiomers of 1,2-propanediol to propionaldehyde.

### SUMMARY

Systems and methods for converting both enantiomers of 1,2-propanediol to propionaldehyde (propanal) are provided. The systems can include engineered organisms, e.g. non-naturally occurring microorganisms. The systems can include cell-free enzyme mixtures. Methods of converting 1-2-propanediol to propanol are provided using the engineered organisms and/or the cell-free enzyme mixtures. The methods can include culturing a microorganism provided herein under conditions and for a period of time sufficient to convert the 1,2-propanediol to propanol. The conditions can include a substantially anaerobic culture medium.

Engineered organisms are provided that convert both enantiomers of 1,2-propanediol to propionaldehyde but do not convert glycerol to 3-hydroxypropionaldehyde. Engineered organisms are provided that convert both enantiomers of 1,2-propanediol to propionaldehyde but do not convert propanal to propanol. In some embodiments the engineered organisms convert both enantiomers of 1,2-propanediol to propanal.

Engineered organisms, e.g. non-naturally occurring microorganisms, are provided containing an exogenous enzyme that converts both enantiomers of 1,2-propanediol to propanal. The microorganism can contain an exogenous diol dehydratase enzyme. The enzyme can be Roseburia inulinivorans diol dehydratase (RiDD) or can be at least 80% identical in protein sequence to RiDD. The microorganism can contain an exogenous diol dehydratase activating enzyme in an amount sufficient to activate the diol dehydratase enzyme. The diol dehydratase activating enzyme can be Roseburia inulinivorans diol dehydratase activating enzyme (RiDD-AE) or can be at least 80% identical in protein sequence to RiDD-AE. The microorganism can further contain an alcohol dehydrogenase enzyme.

The microorganism can contain an exogenous nucleic acid that encodes for one of the enzymes in an amount sufficient to produce the enzyme. The nucleic acid can be a heterologous nucleic acid. The microorganism can be in a substantially anaerobic culture medium. Methods of making the microorganisms can include expressing the nucleic acid that encodes for at least a diol dehydratase in the microorganism; and culturing the microorganism for a suitable period of time and in a suitable culture medium to produce the microorganism. The diol dehydratase, in some embodiments, converts both enantiomers of 1,2-propanediol to propanal.

Cell-free enzyme mixtures are provided containing an isolated diol dehydratase enzyme capable of converting both enantiomers of 1,2-propanediol to propanal; and an alcohol dehydrogenase. The diol dehydratase enzyme can be Roseburia inulinivorans diol dehydratase (RiDD) or can be at least 80% identical in protein sequence to RiDD. The cell-free enzyme mixture can contain an exogenous diol dehydratase activating enzyme in an amount sufficient to activate the diol dehydratase enzyme. The diol dehydratase activating enzyme can be Roseburia inulinivorans diol dehydratase activating enzyme (RiDD-AE) or can be at least 80% identical in protein sequence to RiDD-AE. Methods of making the cell-free enzyme mixtures can include isolating an exogenous enzyme from an engineered organism provided herein.

Methods of converting 1-2-propanediol to propanol, including methods of converting both enantiomers of -2-propanediol to propanol. The methods can include using the engineered organisms and/or the cell-free enzyme mixtures provided herein. The 1,2-propanediol can at least contain (S)-1,2-propanediol. In some embodiments the 1,2-propanediol is a racemic mixture of both enantiomers of the 1,2-propanediol. The methods can include culturing a microorganism provided herein under conditions and for a period of time sufficient to convert the 1,2-propanediol to propanol. The conditions can include a substantially anaerobic culture medium.

### BRIEF DESCRIPTION OF THE DRAWINGS

FIG. 1 depicts a chemical pathway for the enzymatic conversion of both enantiomers of 1,2-propanediol to propanol in a cell-free reactor.

FIG. 2 is a graph of the titer (g/L) of glycerol (squares), propanol (triangles), and propanal (circles) as a function of time (hours) in the hydrogen-driven conversion of a racemic mixture of 1,2-propanediol to propanol in a cell-free reactor under 1 atm of hydrogen.

FIG. 3 depicts a chemical pathway for the conversion of a racemic mixture of 1,2-propanediol to propanol in yeast containing a vector carrying the B12-independent diol dehydratase from R. inulinivorans (RiDD) and the RiDD activating enzyme (RiDD-AE).

FIG. 4 is a graph of the titer (g/L) for ethanol (solid squares), and propanol (solid circles) as a function of time (hours) for yeast harboring an expression plasmid carrying the B12-independent diol dehydratase from R. inulinivorans (RiDD) and the RiDD activating enzyme (RiDD-AE) that were grown on YPD media containing 20 g/L glucose and 20 g/L of a racemic mixture of 1,2-propanediol; No propanol production is observed if the same yeast harboring an expression plasmid carrying RiDD and RiDD-AE are grown in the absence of 1,2-propanediol and no propanol production is observed if yeast harboring an empty expression plasmid are grown in the presence of 20 g/L of a racemic mixture of 1,2-propanediol.

FIG. 5A depicts a chemical pathway describing how the conversion of glycerol, by an activated glycerol dehydratase can be easily monitored using purified DhaT and following the NADH-dependent reduction of 3-hydroxypropionaldehyde to 1,3-propanediol. Similarly, FIG. 5B depicts a chemical pathway for how the conversion of 1,2-propanediol, by an activated diol-dehydratase can be monitored by following the NADH-dependent reduction of propionaldehyde by YADH.

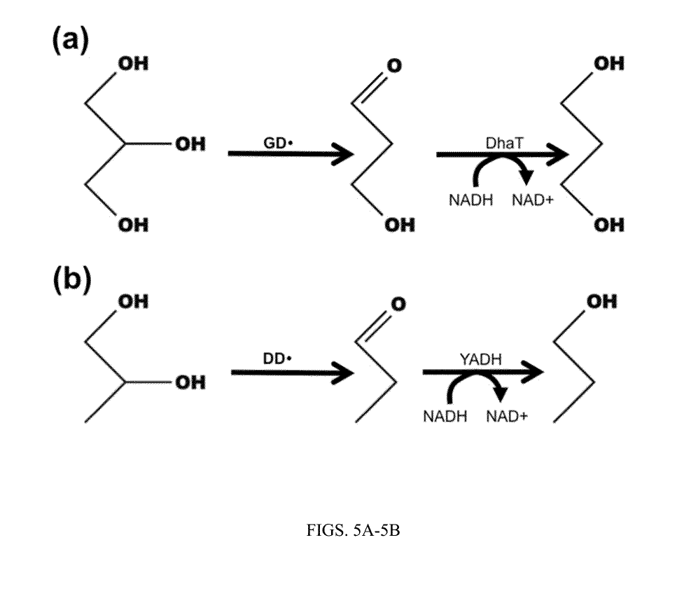

FIGS. 6A and 6B are graphs of a Michaelis-Menton plot showing the specific activity observed for the C. butyricum glycerol dehydratase (CbGD) at different concentrations of (S)-1,2-propanediol (FIG. 6A) or glycerol (FIG. 6B). No activity was observed when (R)-1,2-propanediol was tried as the substrate.

FIG. 7 is a graph of a Michaelis-Menton plot showing the specific activity observed for the B12-independent diol dehydratase from R. inulinivorans (RiDD) at different concentrations of (S)-1,2-propanediol or (R)-1,2-propanediol. No activity was observed when the enzyme was assayed with glycerol as the substrate.

FIG. 8A is a gas chromatograph showing the separation of acetone, propionaldehyde, and 1,2-propanediol. FIG. 8B is a graph of the production of acetone and propionaldehyde (mM) by the B₁₂-independent diol dehydratase from R. inulinivorans (RiDD) as a function of time (seconds). Each assay was performed and samples for GC analysis were prepared as described in the Materials and Methods. Samples were taken and analyzed at the indicted time points. The data in FIG. 8B show the amount of propionaldehyde (closed squares) and acetone (open squares) produced when S-1,2-propanediol was used as substrate as well as the amount of propionaldehyde produced (closed circles) and acetone (open circles) produced when R-1,2-propanediol was used as the substrate for the RiDD.

FIGS. 9A and 9B depict wall-eyed stereoview showing a stick representation of the active site for the R. inulinivorans diol dehydratase (RiDD) alone (FIG. 9A) and aligned with the model of the C. butyricum glycerol dehydratase (CbGD, FIG. 9B). FIG. 9A is a stick representation of residues in the active of the RiDD model and the 2Fₒ-F_{C} composite omit map generated with the simulated annealing protocol with 7% of the model omitted per cycle. FIG. 9B is a stick representation showing a structural overlay of the RiDD model with the CbGD model. This figure depicts the minimum catalytic arrangement of amino acids, the relative position/identity of specific atoms with respect to one another and the orientation of these atoms within the active site of an enzyme capable of converting both enantiomers of 1,2-propanediol to propionaldehyde as described herein.

FIG. 10 depicts two mechanistic pathways that have been proposed for the dehydration of 1,2-propanediol by the R. inulinivorans diol dehydratase (RiDD). Both pathways begin with the abstraction of a hydrogen atom from by a radical on C438 (top left panel). The mechanism is analogous to what has been proposed by Feliks et al. for the CbGD involves the direct dehydration of the hydroxyl group at the second position (bottom left panel). A mechanism that is analogous to what has been proposed for the B₁₂-dependent diol dehydratases involves the migration of both the hydroxyl group and the radical (Top right and bottom left panels).

### DETAILED DESCRIPTION

The majority of commodity chemical precursors for polymer production (propanal, propan-1-ol and/or propan-2-ol, 1,3-propanediol, propylene, or isobutylene) are derived from petrochemical or natural gas sources. As the availability of these sources either diminishes or is used increasing in energy generation, the demand for generation of commodity chemicals from renewables will only increase. Preferably, the renewable feedstocks will not compete with processes used in producing food or a myriad of other environmental issues.

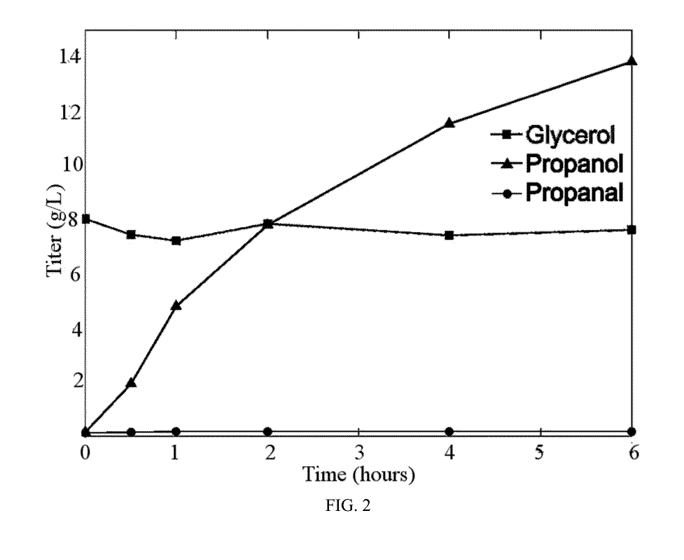

In an embodiment the RiDD catalyst is applied in a cell-free and eukaryotic organism to produce propanol. The use of these catalysts can have the following advantages over existing art and technology; 1) Resistance to glycerol inhibition (FIG. 2), 2) the ability to convert both enantiomers of 1,2-propanediol to propanal and propanol. Embodiments of the present disclose can be performed in a cell free and eukaryotic organism using this enzyme catalyst.

Specifically, embodiments of the present disclosure demonstrate a feasible system for using this enzyme in vitro with other purified enzymes (cell-free reactor) and in yeast (Saccharomyces cerevisiae). The reactor described herein in some embodiments uses hydrogen to quickly reduce the propionaldehyde (propanal) produced by the newly identified diol dehydratase to propanol. Hydrogen drives the complete conversion of both enantiomers of 1,2-propanediol to propanol at high titer with negligible acetone production. We also demonstrate the application of this enzyme in microbial production (fermentation) using recombinant yeast. The application of this bacterial enzyme system, and any glycyl radical enzyme, in a eukaryotic system has not been reported, investigated, or applied in this manner. Absent a mechanism to immediately reduce the propionaldehyde to propanol we also show this new enzyme catalyst can produce both propionaldehyde (propanal) and acetone.

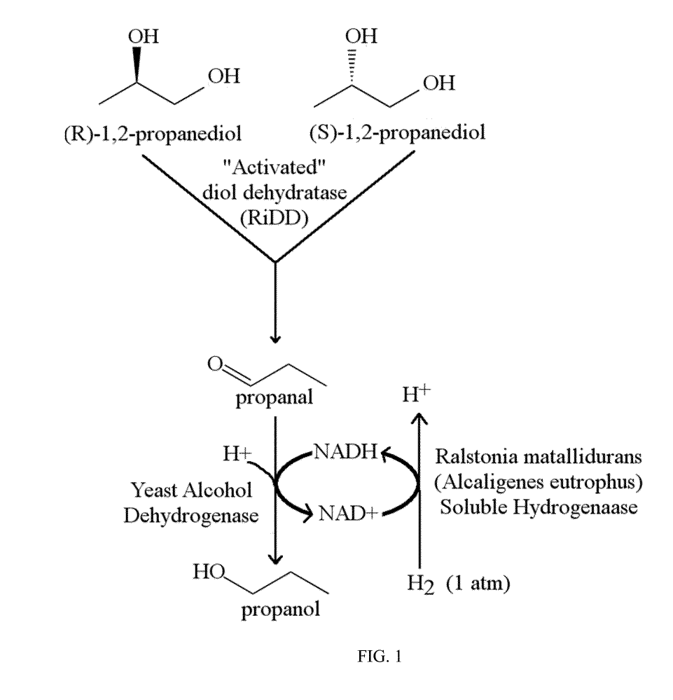

In one embodiment the diol dehydratase can be used in a cell-free system that utilizes hydrogen gas to drive the complete conversion of both enantiomers of 1,2-propanediol all the way to propanol at high titers (FIG. 1 and FIG. 2).

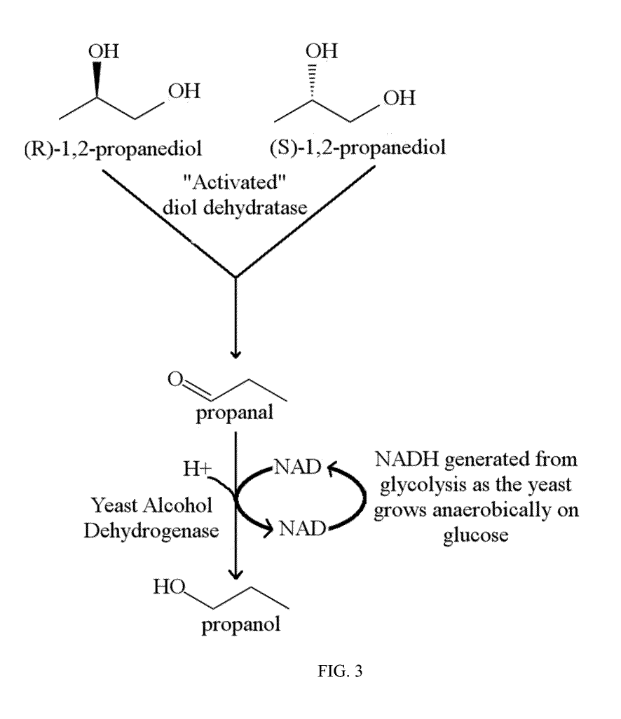

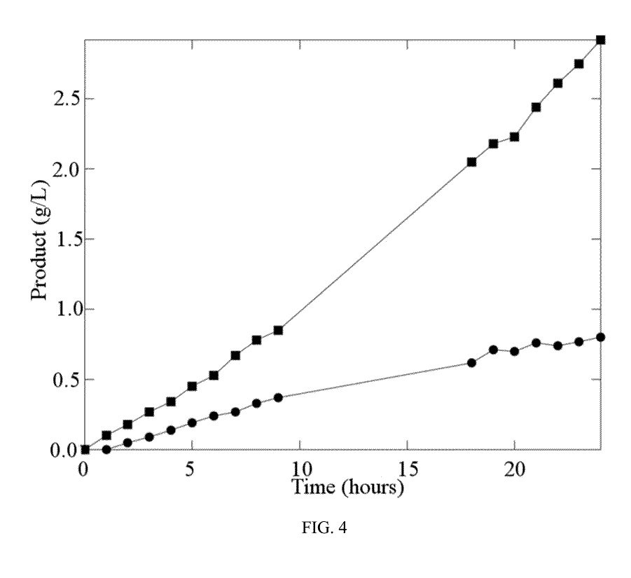

In another particular embodiment the diol dehydratase can be incorporated into yeast to facilitate the production of propanol (FIG. 3 and FIG. 4). In this embodiment, the natural presence of NADH and YADH serves the function of quickly reducing the propionaldehyde to propanol.

In one embodiment the description includes any B₁₂-independent diol dehydratase produced in a microorganism or cell-free system that has the amino acid sequence defined by the protein sequence described below for the RiDD.

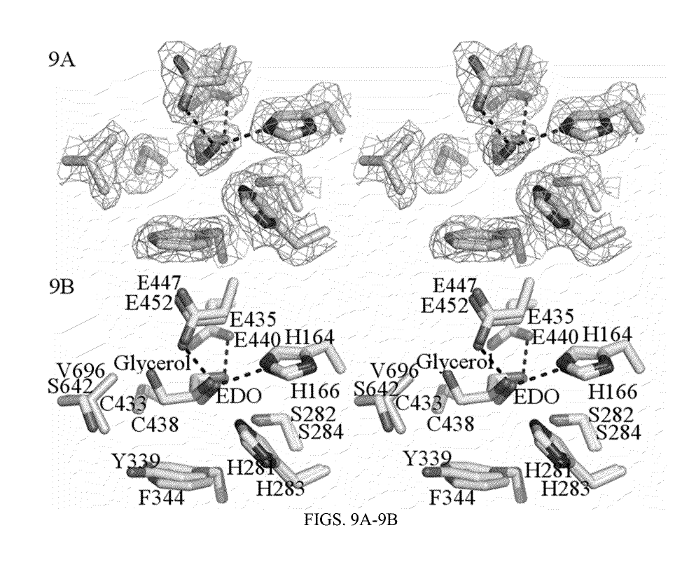

In another embodiment the description includes any protein isolated or expressed in a microorganism that has the active site amino acids spatially arranged as shown in FIG. 9, panels A and B, and as revealed in the atomic coordinates deposited under the PDB ID “4QVO”.

### DEFINITIONS

The term “non-naturally occurring”, when used in reference to a microbial organism or microorganism herein, is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. A metabolic modification refers to a biochemical reaction that is altered from its naturally occurring state. Therefore, non-naturally occurring microorganisms can have genetic modifications to nucleic acids encoding metabolic polypeptides or, functional fragments thereof. Exemplary metabolic modifications are disclosed herein.

The term “isolated”, when used in reference to a microbial organism herein, is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

The terms “microbial organism”, “microorganism”, and “microbe” are used interchangeably herein to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

The terms “1,2-propanediol” and “propanediol” are used interchangeably herein to refer to the di-alcohol having the chemical formula CH₃—CH(OH)—CH₂(OH) and a molecular mass of approximately 76.09 g/mol. 1,2-propanediol may also be known in the art as propylene glycol or propane-1,2-diol. 1,2-propanediol can exist in two possible stereoisomers, (R)-1,2-propanediol and (S)-1,2-propanediol. The commercial 1,2-propanediol exists as a racemic mixture, although enantiomerically pure optical isomers can be obtained. As used herein, 1,2-propanediol should be understood to include any ratio of the stereoisomers, for example from 100% (R)-1,2-propanediol to 100% (S)-1,2-propanediol, from 99% (R)-1,2-propanediol to 99% (S)-1,2-propanediol, from 90% (R)-1,2-propanediol to 90% (S)-1,2-propanediol, or others.

The terms “propanol” and “n-propanol” are used interchangeably herein to mean a primary alcohol with the molecular formula of C₃H₈O and a molecular mass of 60.1 g/mol. N-propanol is also known in the art as 1-propanol, 1-propyl alcohol, n-propyl alcohol, or propan-1-ol.

The terms “propanal” and “propionaldehyde” are used interchangeably herein to refer to the saturated 3-carbon aldehyde having the chemical formula CH₃—CH₂—CHO and a molecular mass of approximately 58.08 g/mol.

The terms “glycerol”, “glycerine”, and “glycerin” are used interchangeably herein to refer to the 1,2,3-tri-alcohol or propane having the chemical formula CH₂(OH)—CH(OH)—CH₂(OH) and a molecular mass of approximately 92.09 g/mol. Glycerol is also known as propane-1,2,3-triol, propanetriol, or 1,2,3-trihydroxypropane.

The terms “3-hydroxypropionaldehyde” and 3-hydroxypropanal” are used interchangeably herein to refer to the molecule having the chemical formula COH—CH₂—CH₂(OH) and a molecular mass of approximately 74.08 g/mol. 3-hydroxypropionaldehyde is also known as 3-oxo-1-propanol, hydroxypropanal, β-hydroxypropanal, or reuterin.

The term “substantially anaerobic”, when used in reference to a culture or growth condition herein, is intended to mean that the amount of oxygen is less than about 15%, less than about 10%, less than about 5%, or less than about 3% of saturation for dissolved oxygen in the liquid media The term “substantially anaerobic” is also intended to include sealed chambers of liquid or solid medium maintained with an atmosphere of less than about 5%, 3%, 2%, or 1% oxygen.

The term “exogenous” as it is used herein is intended to mean that the referenced molecule or the referenced activity is introduced into the host microorganism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microorganism. When used in reference to a biosynthetic activity, the term refers to an activity that is introduced into the host reference microorganism. The source can be, for example, a homologous or heterologous encoding nucleic acid that expresses the referenced activity following introduction into the host microorganism. The term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microorganism. Accordingly, exogenous expression of an encoding nucleic acid can utilize either or both a heterologous or homologous encoding nucleic acid.

Non-naturally occurring microorganisms can contain stable genetic alterations, which refers to microorganisms that can be cultured for greater than about 3, 4, 5, 6, 8, or 10 generations without loss of the alteration. Particularly stable modifications will persist more than about 25 generations, and more particularly, stable genetic modifications will be greater than 50 generations, including indefinitely.

The genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

The term “cell-free”, as used herein, refers to a reactor or an enzyme mixture having been separated from any cells, including the cells that secreted the enzymes. Cell-free reactors and cell-free enzyme mixtures can be prepared by any of a variety of methodologies that are known in the art, such as filtration or centrifugation methodologies. In some embodiments, the reactor or the enzyme mixture can be, for example, partially cell-free, substantially cell-free, or entirely cell-free.

The term “cell-free reactor” refers to any reactor vessel, of any construct (glass, steel, wood, etc.) that can be made anaerobic (free of molecular oxygen) and designed to contain only the enzymatic of catalytic components and or any and all of the chemicals required for the function and maintenance of the catalytic components and enzymes required to perform the conversion of both enantiomers of 1,2-propanediol to propionaldehyde and/or propanol. The reaction vessel can have any number of access ports for addition/extraction of liquids (from the liquid volume) or gases (from the head-space or gas volume), so long as functioning enzymes/catalysts are maintained.

In the “cell-free reactor” described herein, the specific enzymes and biological catalysts and chemical cofactors for the continuous conversion of both enantiomers of 1,2-propanediol includes; an “appropriate buffer” at or within 1.0, 0.8, or 0.5 pH units of pH 7.0, the diol dehydratase (RiDD) or any homologue thereof that brings the appropriate amino acid sides chains into the atomic arrangement described herein, the RiDD activating enzyme (RiDD-AE), yeast alcohol dehydratase (YADH) or any NADH- or NADPH-dependent dehydrogenase capable of reducing propionaldehyde to propanol, the “uptake hydrogenase” from Alcaligenes eutrophus H16 (a.k.a. Ralstonia eutrophus and Ralstonia metallidurans), the appropriate nicotinamide cofactor (in this embodiment nicotinamide, adenine dinucleotide), and 1 atm of hydrogen gas.

In the simplest embodiment the “cell free reactor” would include only “catalytic” amounts of the enzymes described herein. Specifically, a catalytic amount is one molecule and anything greater than one enzyme molecule. In the data shown in FIG. 2 of this disclosure the cell free reactor includes the following; 10 μM of RiDD, 100 μM yeast alcohol dehydrogenase (YADH), and 200 μM of the soluble hydrogenase from Ralstonia metallidurans (formally Alcaligenes eutrophus). The reaction was carried out under strictly anaerobic conditions (oxygen-free argon gas) and initiated by the addition of the reduced RiDD-AE (50 μM final) and S-adenosylmethionine (1 μM). 100% hydrogen gas was maintained in the head space of the reactor at 1 atm and the reaction also included 1 mM NADH, 20 mM TRIS pH 7.9, and 1% glycerol. The glycerol was added in order to further confirm two aspects described herein, specifically that the dehydratase does not utilize glycerol as a substrate nor is the dehydratase inhibited by glycerol.

### Enzymes

Diol dehydratase enzymes are provided herein. In some embodiments, an enzyme is provided that can convert both (S)-1,2-propanediol and (R)-1,2-propanediol into propanal (FIG. 1). The enzyme can be a glycyl radical enzyme (GRE). GREs represent a large superfamily of enzymes capable of catalyzing many difficult and diverse chemical reactions¹. As the name implies, all of these enzymes utilize a radical mechanism and function under strictly anaerobic conditions. The current list of chemical transformations catalyzed by GREs now includes the formation of C—C bonds (benzyl succinate synthase), C—C bond cleavage (PFL and 4-hydroxyphenylacetate decarboxylase)³, ribonucleotide reduction (ARNR)⁴, dehydration reactions (glycerol dehydratase), and C—N bond cleavage (choline TMA-lyase)⁶. Among the reactions catalyzed by GREs, the B₁₂-independent dehydratases represent a viable commercial catalyst in the production of a number of commodity chemicals. Similar to the dehydration reactions, the observation of a GRE that performs C—N bond cleavage by Craciun et al. has further extended the parallel chemistry being catalyzed by GREs and their B₁₂-dependent counterparts.

In general, most GREs are homo-dimers with a subunit size of less than 100 kDa. The RiDD structure described here is a ten-stranded β-barrel structure, originally identified in the class III ribonucleotide reductases. Moreover, the overall structure of the RiDD aligns very nicely with the CbGD we reported previously (RMSD of 0.6 Å for the backbone α-carbon atoms). Comparison of the RiDD structure with the CbGD structure reveals an addition domain that is due to a 48 amino acid insert that is not present in the CbGD. The domain extends a helix found in the CbGD and includes another helix, loop, and helix. Looking at GRE structure more broadly, the highest degree of structural homology between the RiDD and other GREs appears to be in the C-terminal domain of the RiDD. This is not unexpected given that the C-terminal domain contains the site of glycyl radical formation. Of particular significance, is the relative spatial orientation of the key glycine residue relative to the side chains of other conserved amino acids. Similar to the early observations, the glycine residue is found within a loop that folds into the protein and over the core barrel structure⁷. For the RiDD dehydratase enzyme described herein, these amino acids are C438, R814, G817, and Y818. Among these amino acids, Y818 is the only residue that is not conserved, as this residue is a phenylalanine in 4-HPAD. However, the spatial arrangement of these residues is identical in all of the GREs for which we have structural information, and therefore implies a conserved or universal function. A reasonable hypothesis is that these residues have a role in facilitating the delocalization or sharing of the radical (via hydrogen atom exchange) between the alpha carbon of the conserved glycine residue and the sulfur atom of the conserved cysteine residue. It will be recognized by those skilled in the art there are a myriad of approaches for both the genomic recombination of the genes related to this disclosure as well as introduction of the genes via vectors with appropriate expression elements. It is clearly defined herein how the catalytic amino acids are oriented in space that allows the enzyme to function. Those skilled in the art will recognize that, due to the universal genetic code, numerous nucleic acid sequences could encode a protein substantially identical in polypeptide sequence, structure, or function defined herein. In one embodiment the disclosure provided herein is intended to cover any nucleic acid sequence that translates into a protein with the active amino acids spatially arranged as shown in FIG. 9A.

In one embodiment, the atoms of the substrate (both R- and S-1,2-propanediol) are bound in an active site such as to interact within the parameters implied in FIG. 9A and FIG. 9B and the following general description; the oxygen atom of the hydroxyl group at the first (#1) position of 1,2-propanediol will be bound in the active site within 2.8 angstroms of a side chain oxygen atoms of E440 and within 2.9 angstroms of the side chain oxygen atom of 5284 and within 3.2 angstroms of a side chain nitrogen atom of H166; the first (#1) carbon atom of 1,2-propanediol will be located in the active site within 3.8 angstrom of the side chain sulfur atom of C438 and within 3.6 angstroms of the side chain oxygen atom of Y694; the oxygen atom of the hydroxyl group at the second (#2) position of 1,2-propanediol is located within 2.7 angstroms of a side chain oxygen atom from D452 and within 2.9 angstroms of a side chain nitrogen atom of H166 and 2.9 angstroms of a side chain nitrogen atom of H283. These residues appear to be strictly conserved in the family of B₁₂-independent dehydratases and experts in the field will recognize that the numbering will change for each of the homologous residues identified here. However, their atomic arrangement will be preserved for the dehydration of both enantiomers. Amino acids specific to the functionality associated with utilizing both enantiomers of 1,2-propanediol include the amino acids V696 and F344. These position and lack of any hydrogen bonding between these amino acids within the active site allows for multiple orientations of 1,2-propanediol while maintaining the interactions described above and, hence, the ability to utilize both enantiomers. The arrangement described herein and in the atomic coordinates (PDB ID 4QVO) is specific for the function described herein.

In some embodiments the enzyme is selective for propanediol. In particular, in some embodiments the enzyme will accept both enantiomers of 1,2-propanediol but will not act accept glycerol as a substrate. In some embodiments the enzyme is Roseburia inulinivorans diol dehydratase (RiDD). In some embodiments the enzyme has a sequence according to SEQ ID NO:5 described below. In some embodiments the enzyme has a sequence with at least about 80%, at least about 85%, at least about 90%, at least about 95%, or at least about 99% similarity to SEQ ID NO:5. The enzyme can be used in microorganisms and cell-free reactors, as described below, for the conversion of both enantiomers of 1,2-propanediol to propanal and a process to produce products that include propanal and propanol (See FIGS. 1-4, FIG. 7, and FIG. 8).

Also provided is a nucleic acid (gene) that, when expressed, the gene codes for a B₁₂-independent dehydratase in the organism Roseburia inulinivorans. This gene can be successfully expressed using the translational machinery in Escherichia coli as well as Saccharomyces cerevisiae, as described below. In both cases, the diol dehydratase encoded by this gene can convert both enantiomers of 1,2-propanediol into propanal. In some embodiments, the enzyme will not convert, nor is the activity inhibited by the presence of glycerol.

In some embodiments a nucleic acid sequence is provided that encodes for a diol dehydratase. In one embodiment the nucleic acid sequence encoding the diol dehydratase is defined in NCBI database accession #WP_{—}007885173; version WP_{—}007885173 GI:495160372 in R. inulinivorans currently annotated as “pyruvate formate lyase”. This annotation is clearly incorrect and heretofore we refer to the B₁₂-independent diol dehydratase from R. inulinivorans as “RiDD”. The diol dehydratase activating enzyme gene is defined by the nucleic acid sequence defined in NCBI database accession #ABC25540; version ABC25540.1 GI:83596383. We heretofore refer to this enzyme as the “RiDD-AE”. In another embodiment these nucleic acid sequences can be synthesized de novo and optimized for codon usage in either Saccharomyces cerevisiae or Escherichia coli. In some embodiments nucleic acids are provided that encode for a diol dehydratase and have a sequence with at least about 80%, at least about 85%, at least about 90%, at least about 95%, or at least about 99% similarity to SEQ ID NO:1 or SEQ ID NO: 3. In some embodiments nucleic acids are provide having a sequence according to SEQ ID NO:1 or SEQ ID NO:3, i.e. they are 100% identical to SEQ ID NO:1 or SEQ ID NO:3. In some embodiments the diol dehydratase activating enzyme has a sequence with at least about 80%, at least about 85%, at least about 90%, at least about 95%, or at least about 99% similarity to SEQ ID NO:6. In some embodiments the diol dehydratase activating enzyme has a sequence according to SEQ ID NO:6, i.e. it is 100% identical to SEQ ID NO:6.

In another embodiment the nucleic acid sequence encoding the soluble hydrogenase subunits for the nickel-iron hydrogenase from Ralstonia metallidurans or Ralstonia eutrophus (formally Alcaligenes eutrophus) and all the accessory genes required for assembly of the functional enzyme. There is considerable existing art with respect to the application of the soluble hydrogenase from R. eutrophus H16 for the regeneration of the NADH cofactor. However, this enzyme has not been used previously in the production of propanol from both enantiomers of 1,2-propanediol.

In one embodiment, the nucleic acid contains a sequence encoding one or more of the enzymes described herein that when translated into protein in a microorganism (in vivo) or in a cell-free environment (in vitro) results in an enzyme capable of catalyzing the chemical conversions of both enantiomers of 1,2-propanediol to propanal. Expressed in a microorganism or in a cell-free environment, the system can allow for the following; conversion of both enantiomers of 1,2-propanediol to propanal; conversion of propanal into propanol; and/or oxidation of hydrogen gas coupled to reduction of NAD+ to NADH. In one particular embodiment the microorganism expressing these genes can have the capability to produce propanal or propanol from other sugar sources. In another embodiment the cell-free system will allow for the direct conversion of a racemic or enantiomeric (either “R” or “S”) solution of 1,2-propanediol at high titers (100 g/L or higher) to propanol under 1 atm of hydrogen gas.

Described herein are nucleic acids or nucleic acid constructs that contain one or more of the nucleic acids used for generating the respective enzyme catalysts in vitro or in generating a recombinant microorganism described below. Where appropriate, this is any genetic sequence that universally translates into the protein sequence of the enzyme catalyst described herein.

In one embodiment, the nucleic acid can be a nucleic acid contained in a plasmid or vector. In another embodiment the nucleic acid can be incorporated or recombined as part of the host genome or exist in that genome as part of the native organism with the intention that the natural activity of the gene product provides a function described herein (i.e. yeast alcohol dehydrogenase will catalyze the NADH-dependent reduction of propionaldehyde to propanol).

The nucleic acid sequences code for amino acid sequences that fold into enzymes capable of performing the chemical transformations described herein. Their sequences and, where appropriate, their three dimensional structures have been determined at the atomic level are described herein or in some cases may be found online at the NCBI or GenBank as mentioned earlier.

The disclosure applies to the diol dehydratase, aldehyde reductase, and hydrogenase enzymes as products of the gene described in the proceeding section. For the diol dehydratase the disclosure described herein applies the “wild-type” enzyme(s) in in their isolated (cell-free) or as expressed in a microorganism, in this case yeast.

Specifically, the relative orientations of an active site for dehydration of R- and S-1,2-propanediol can be as follows, the substrate (R- or S-1,2-propanediol) are bound in an active site such as to interact within the parameters or atoms shown in FIG. 9A and FIG. 9B, detailed in the coordinate file (PDB ID “4QVO”), and the following general description; the oxygen atom of the hydroxyl group at the first (#1) position of 1,2-propanediol will be bound in the active site within 2.8 angstroms of a side chain oxygen atoms of E440 and within 2.9 angstroms of the side chain oxygen atom of 5284 and within 3.2 angstroms of a side chain nitrogen atom of H166; the first (#1) carbon atom of 1,2-propanediol will be located in the active site within 3.8 angstrom of the side chain sulfur atom of C438 and within 3.6 angstroms of the side chain oxygen atom of Y694; the oxygen atom of the hydroxyl group at the second (#2) position of 1,2-propanediol is located within 2.7 angstroms of a side chain oxygen atom from D452 and within 2.9 angstroms of a side chain nitrogen atom of H166 and 2.9 angstroms of a side chain nitrogen atom of H283. These residues appear to be strictly conserved in the family of B₁₂-independent dehydratases and experts in the field will recognize that the numbering will change for each of the homologous residues identified here. However, their atomic arrangement can be preserved for the dehydration of both enantiomers. Amino acids specific to the functionality associated with utilizing both enantiomers of 1,2-propanediol include the amino acids V696 and F344. These position and lack of any hydrogen bonding between these amino acids within the active site allows for multiple orientations of 1,2-propanediol while maintaining the interactions described above and, hence, the ability to utilize both enantiomers. The arrangement described herein and in the atomic coordinates (PDB ID 4QVO) is specific for the function described herein.

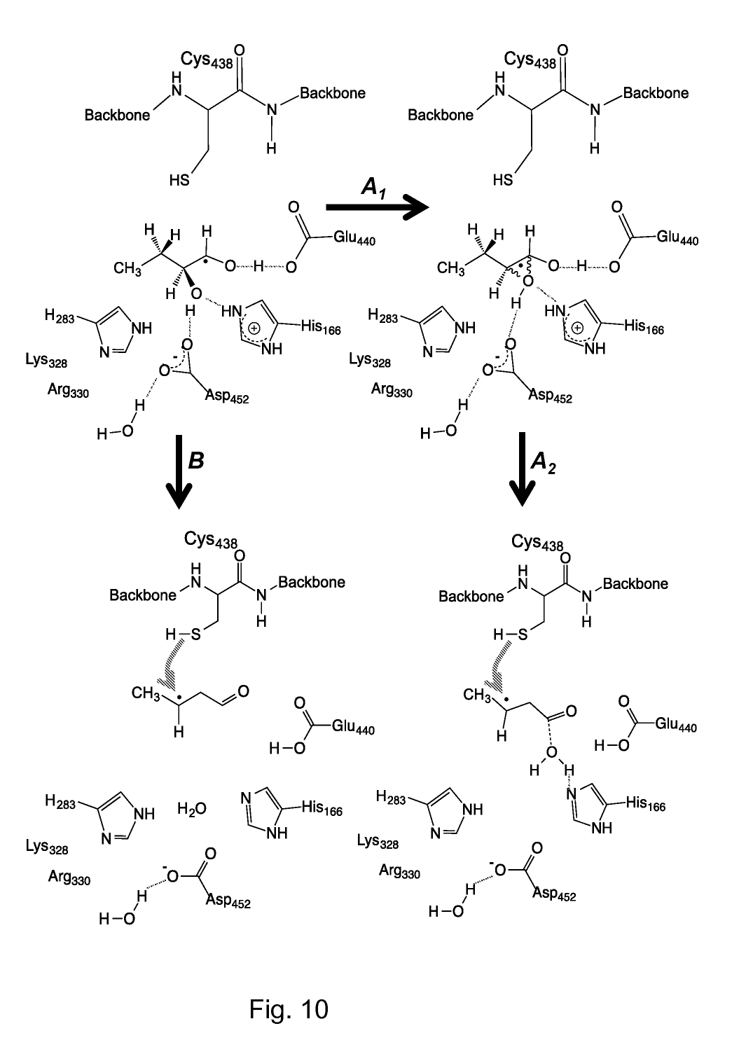

In another embodiment the description refers to any enzyme or protein with the amino acids that are spatial oriented (as described herein) and used for the conversion of both enantiomers of 1,2-propanediol to propanal and subsequently propanol in the organism Saccharomyces cerevisiae. Again, while the specific sequence numbering may vary, the relative orientation of the substrate (R- or S-1,2-propanediol) atoms and the atoms of the catalytic amino acids will be maintained as described in the proceeding section. The arrangement of these atoms facilitates the specific dehydration for both enantiomers via one of two proposed mechanisms outlined in FIG. 10.

### Non-Naturally Occurring Microorganisms Containing Diol Dehydratase Enzymes

Non-naturally occurring microorganisms are provided containing one or more of the enzymes described herein. In some embodiments the non-naturally occurring microorganisms convert both enantiomers of 1,2-propanediol into propanal. In some embodiments the non-naturally occurring microorganisms further convert the propanal into propanol. The non-naturally occurring microorganisms can convert both enantiomers of 1,2-propanediol, but will not convert glycerol, into propanal or propanol.

The non-naturally occurring microorganisms can be prepared by introducing one or more exogenous genes that encode for an enzyme described herein or by introducing a vector containing a nucleic acid encoding enzyme described herein. In particularly useful embodiments, exogenous expression of the encoding nucleic acids is employed. Exogenous expression confers the ability to custom tailor the expression and/or regulatory elements to the host and application to achieve a desired expression level that is controlled by the user. However, endogenous expression also can be utilized in other embodiments such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

Host microbial organisms can be selected from, and the non-naturally occurring microbial organisms generated in, for example, bacteria, yeast, fungus or any of a variety of other microorganisms applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger and Pichia pastoris. E. coli is a particularly useful host organism since it is a well characterized microbial organism suitable for genetic engineering. Other particularly useful host organisms include yeast such as Saccharomyces cerevisiae.

Exogenous nucleic acid sequences encoding an enzyme described herein can be introduced stably or transiently into a host cell using techniques well known in the art including, but not limited to, conjugation, electroporation, chemical transformation, transduction, transfection, and ultrasound transformation. For exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

In a further aspect of each of the above embodiments, the exogenous nucleic acid can be a heterologous nucleic acid. In some embodiments, the non-naturally occurring microbial organism is in a substantially anaerobic culture medium.

### Cell-Free Reactors Containing Diol Dehydratase Enzymes

The enzymes described herein can be isolated and used in a cell-free reactor for the conversion of both enantiomers of 1,2-propanediol to propanal. The enzymes can be combined with an alcohol dehydrogenase to convert the propanal into propanol. In some embodiments the cell-free reactor converts 1,2-propanediol to propanol with high titers, e.g. greater than 10 g/L, greater than 25 g/L, greater than 50 g/L, or even greater than 100 μL. In some embodiments high titers are achieved when the reaction is performed under anaerobic conditions, e.g. under an oxygen-free hydrogen gas.

### EXAMPLES

### Codon Optimization and Amplification of Genes for RiDD and RiDD-AE

The parent plasmids from which all genes were manipulated further were created as follows. The genes for the Roseburia inulinivorans diol dehydratase (RiDD) and RiDD activating enzyme (RiDD-AE), NCBI accession #'s WP_{—}007885173 and ABC25540 respectively, were codon-optimized for expression in E. coli by de novo gene synthesis. The genes for RiDD and RiDD-AE, NCBI accession #'s WP_{—}007885173 and ABC25540 respectively, were codon-optimized for expression in E. coli and cloned into the commercially available pTRCHis™ vector in order to introduce a 6× polyhistidine tail at the N-terminus with the following sequence “MSHHHHHGSG”. The enzyme YqhD is native to E. coli and was cloned directly into the same vector after PCR amplification from crude extract. The genes that were codon optimized were those corresponding to the RiDD and the RiDD-AE. Codon optimization was performed

The RiDD gene sequence optimized for expression in S. cerevisiae is:

(SEQ ID NO: 1)
ATGGGTAACTATGATTCAACTCCAATTGCAAAGTCAGATAGAATTAAGAG
ATTAGTTGACCACTTATATGCTAAAATGCCAGAAATAGAGGCAGCAAGGG
CTGAATTAATAACAGAATCTTTTAAAGCAACAGAGGGACAGCCTGTTGTT
ATGAGGAAAGCCAGGGCCTTCGAACATATTTTAAAGAATTTGCCTATTAT
TATTAGACCAGAAGAATTGATCGTTGGTTCAACTACAATTGCACCAAGAG
GATGTCAAACATATCCTGAATTTTCATACGAATGGTTAGAGGCCGAATTT
GAAACTGTTGAGACCAGATCAGCAGACCCATTCTATATTTCAGAGGAAAC
AAAGAAGAGATTATTGGCAGCAGATGCATATTGGAAGGGTAAAACAACAT
CTGAATTGGCTACCTCATATATGGCCCCTGAAACCTTGAGAGCTATGAAG
CATAACTTCTTTACACCAGGAAACTACTTCTATAATGGTGTTGGACATGT
TACTGTCCAGTACGAAACTGTCTTGGCTATCGGTTTGAATGGAGTCAAGG
AGAAAGTTAGAAAAGAAATGGAGAACTGCCATTTTGGTGATGCAGACTAT
TCAACTAAAATGTGCTTCTTAGAGTCTATTTTAATTTCTTGTGACGCTGT
TATAACCTATGCCAACAGATACGCTAAAATGGCCGAGGAAATGGCTGAAA
AGGAGACAGATGCAGCTAGAAGACAGGAATTGTTGACAATTGCAAGAGTT
TGTAAGAACGTCCCTGAATTTCCTGCCGAATCTTTCCAAGAAGCCTGTCA
ATCTTTCTGGTTTATACAACAAGTCTTGCAAATAGAGTCTTCTGGTCATT
CTATTTCTCCAGGTAGATTTGATCAATACATGTACCCATATTATGAGAAA
GATTTGAAGGAGGGTTCATTGACTAGGGAATATGCACAAGAGTTGATAGA
TTGTATATGGGTTAAGTTGAACGACTTAAACAAATGCAGGGACGCCGCTT
CTGCTGAAGGTTTTGCAGGTTATTCATTGTTCCAGAATTTGATAGTCGGT
GGTCAAACAGTACAGGGTAGAGATGCAACCAATGACTTGTCTTTCATGTG
TATCACTGCCTCAGAACACGTCTTCTTACCAATGCCTTCATTATCTATTA
GAGTTTGGCACGGTTCTTCAAAGGCCTTGTTGATGAGAGCCGCAGAATTG
ACCAGAACAGGAATCGGATTGCCAGCTTATTACAATGATGAAGTTATTAT
ACCAGCTTTGGTTCATAGAGGTGCTACAATGGACGAAGCAAGAAATTACA
ATATAATAGGATGTGTTGAGCCTCAAGTTCCAGGTAAAACAGATGGATGG
CATGATGCTGCTTTCTTTAATATGTGTAGACCTTTAGAAATGGTCTTTTC
AAACGGTTACGACAACGGTGAAATAGCCTCAATTCAGACTGGTAATGTTG
AATCATTTCAGTCATTTGATGAGTTCATGGAAGCCTACAGGAAACAAATG
TTATATAATATTGAATTGATGGTCAATGCTGACAACGCCATTGACTACGC
TCACGCCAAATTGGCCCCTTTGCCTTTCGAGTCTTGTTTGGTCGATGATT
GTATTAAGAGAGGAATGTCAGCACAAGAAGGTGGTGCAATATACAATTTT
ACCGGTCCACAAGGATTCGGTATTGCTAATGTAGCTGATTCTTTGTATAC
CATAAAGAAGTTGGTATTTGAAGAAAAGAGAATTACCATGGGTGAGTTGA
AGAAGGCCTTGGAAATGAACTACGGAAAGGGATTGGACGCAACAACAGCA
GGAGACATTGCTATGCAGGTCGCAAAGGGTTTGAAAGACGCCGGTCAAGA
AGTCGGACCTGATGTCATAGCTAATACTATTAGACAAGTTTTGGAAATGG
AATTGCCTGAGGATGTCAGGAAGAGATATGAGGAAATTCATGAGATGATA
TTGGAATTACCAAAATACGGTAATGATATTGACGAAGTTGACGAATTAGC
TAGAGAAGCCGCATACTTTTATACCAGGCCATTAGAAACATTCAAGAACC
CAAGGGGAGGTATGTATCAAGCAGGTTTGTACCCAGTCTCTGCCAATGTT
CCTTTAGGTGCACAAACCGGTGCAACACCAGACGGTAGATTGGCCCATAC
ACCTGTTGCAGATGGAGTAGGTCCAACATCAGGATTTGATATTTCTGGAC
CAACTGCCTCTTGTAACTCTGTCGCCAAATTAGATCATGCTATTGCATCT
AACGGTACCTTGTTTAATATGAAGATGCATCCTACTGCTATGGCCGGAGA
AAAGGGTTTGGAATCTTTCATCTCTTTAATTAGAGGTTATTTTGATCAAC
AGGGAATGCACATGCAATTCAATGTTGTTGACAGGGCCACTTTATTAGAT
GCTCAAGCTCACCCTGAGAAGTACTCTGGATTGATTGTTAGGGTAGCAGG
TTATTCTGCTTTATTCACCACCTTATCTAAGTCTTTACAAGATGATATAA
TCAAGAGGACAGAACAAGCTGACAATAGATAA

The RiDD-AE gene optimized for expression in S. cerevisiae is:

(SEQ ID NO: 2)
ATGAAGGAATATTTGAATACCTCTGGAAGAATTTTCGATATACAGAGGTA
CTCTATTCATGATGGACCTGGTGTAAGGACAATAGTATTCTTGAAGGGAT
GTGCTTTAAGGTGCAGATGGTGCTGCAATCCAGAGTCACAATCATTTGAA
GTTGAAACAATGACAATTAATGGTAAGCCTAAGGTTATGGGTAAAGACGT
CACAGTAGCCGAAGTTATGAAAACCGTTGAAAGAGATATGCCATATTACT
TACAATCAGGTGGTGGTATTACCTTATCAGGAGGAGAATGCACATTACAA
CCAGAATTTTCATTGGGATTATTGAGGGCTGCTAAAGACTTAGGTATCTC
TACTGCCATAGAATCAATGGCCTACGCTAAGTATGAAGTCATCGAAACTT
TGTTGCCATACTTGGACACCTACTTAATGGATATTAAACACATGAACCCA
GAAAAGCATAAGGAGTACACTGGACATGATAACTTAAGGATGTTGGAGAA
CGCCTTAAGAGTCGCACACTCTGGTCAAACTGAATTAATTATTAGGGTCC
CTGTTATCCCAGGTTTCAACGCTACAGAACAAGAATTATTAGATATTGCC
AAGTTCGCCGATACTTTACCAGGTGTAAGGCAGATACACATCTTGCCTTA
TCACAATTTCGGTCAAGGAAAATACGAAGGTTTGAATAGAGATTACCCTA
TGGGTGATACTGAAAAGCCTTCTAATGAACAGATGAAGGCCTTTCAAGAA
ATGATACAAAAGAATACTTCATTGCATTGCCAAATCGGTGGTTAA

The RiDD gene optimized for expression in E. coli is:

(SEQ ID NO: 3)
ATGGGTAATTATGATTCAACGCCAATAGCCAAAAGCGATCGTATTAAACG
CTTAGTTGACCACCTGTACGCCAAGATGCCGGAAATCGAGGCCGCACGCG
CTGAGCTGATCACGGAGAGTTTTAAAGCCACCGAGGGGCAGCCTGTCGTT
ATGCGTAAGGCACGTGCGTTTGAACACATTTTAAAGAACCTGCCAATCAT
TATACGTCCTGAAGAACTGATTGTTGGAAGCACTACGATCGCCCCTCGCG
GGTGTCAGACCTATCCGGAGTTTAGTTATGAATGGCTGGAAGCTGAGTTT
GAGACTGTTGAAACTCGCTCTGCAGATCCATTCTATATTTCTGAGGAAAC
GAAGAAACGCCTGCTGGCCGCGGATGCTTATTGGAAAGGCAAAACTACTT
CTGAACTGGCTACCTCTTATATGGCACCGGAAACACTGCGCGCAATGAAG
CATAATTTCTTTACCCCAGGTAATTATTTCTATAATGGCGTAGGCCATGT
GACGGTGCAGTATGAAACGGTACTGGCAATTGGCCTGAATGGAGTAAAAG
AGAAGGTACGTAAAGAGATGGAAAACTGTCATTTTGGTGATGCAGACTAC
AGCACTAAGATGTGTTTCCTGGAGTCAATTCTGATTAGTTGTGATGCTGT
TATAACTTATGCGAATCGTTATGCCAAAATGGCAGAAGAAATGGCAGAAA
AGGAAACCGACGCAGCCCGTCGCCAGGAACTGCTGACCATAGCGCGTGTA
TGTAAGAACGTACCTGAATTTCCTGCAGAATCTTTTCAGGAGGCTTGTCA
ATCATTTTGGTTCATTCAGCAGGTCTTACAGATCGAGAGTTCAGGGCATT
CAATCAGCCCAGGCCGCTTTGACCAGTATATGTATCCGTATTATGAGAAG
GACCTGAAGGAAGGTTCACTGACCCGCGAATATGCACAGGAACTGATTGA
TTGCATTTGGGTTAAACTGAATGATCTGAATAAGTGCCGCGACGCAGCGT
CAGCCGAGGGCTTTGCGGGTTATAGCCTGTTTCAAAATCTGATCGTTGGT
GGACAGACTGTTCAAGGTCGTGATGCCACTAACGACCTGAGCTTTATGTG
TATTACTGCAAGTGAGCACGTGTTCCTGCCGATGCCGAGCTTATCAATTC
GCGTTTGGCACGGCTCAAGCAAGGCTTTACTGATGCGCGCCGCTGAGCTG
ACACGCACAGGTATTGGCCTGCCGGCCTATTATAATGATGAAGTCATTAT
ACCTGCCCTGGTTCATCGTGGTGCAACAATGGATGAGGCGCGCAACTATA
ACATTATCGGTTGTGTGGAACCACAGGTACCTGGGAAAACCGATGGTTGG
CACGACGCGGCGTTCTTTAATATGTGTCGTCCGCTGGAGATGGTTTTCAG
TAATGGCTACGATAATGGGGAGATTGCAAGCATTCAGACTGGCAATGTGG
AGTCTTTCCAGTCATTTGACGAGTTTATGGAAGCCTACCGTAAACAAATG
CTGTATAATATAGAGCTGATGGTTAATGCAGATAATGCTATAGATTACGC
ACATGCCAAATTAGCCCCGCTGCCGTTTGAAAGCTGTCTGGTAGATGATT
GCATTAAACGCGGTATGAGCGCCCAGGAAGGCGGCGCGATATATAATTTC
ACGGGTCCACAGGGGTTTGGTATAGCAAACGTTGCAGATAGCCTGTACAC
AATAAAGAAATTAGTGTTCGAAGAAAAGCGCATCACAATGGGAGAACTGA
AGAAAGCACTGGAAATGAATTATGGAAAGGGGTTAGATGCAACAACCGCG
GGTGACATTGCAATGCAGGTCGCCAAAGGGCTGAAGGACGCGGGACAGGA
GGTAGGTCCGGACGTCATCGCGAATACCATTCGTCAGGTGCTGGAAATGG
AACTGCCGGAAGATGTTCGTAAACGCTATGAAGAAATTCACGAGATGATT
CTGGAATTACCGAAATATGGCAACGATATAGATGAGGTGGATGAACTGGC
CCGCGAAGCGGCCTACTTTTATACCCGCCCTCTGGAAACGTTTAAGAATC
CGCGCGGCGGTATGTATCAGGCGGGTCTGTACCCGGTGAGTGCCAATGTG
CCACTGGGAGCACAAACGGGTGCGACGCCTGATGGTCGCCTGGCGCACAC
GCCAGTAGCGGATGGAGTTGGTCCTACTTCTGGGTTTGATATATCTGGTC
CGACCGCATCATGCAATTCAGTTGCGAAACTGGATCATGCCATCGCAAGC
AACGGCACCCTGTTCAATATGAAAATGCACCCTACCGCGATGGCAGGAGA
AAAGGGTCTGGAGAGTTTCATAAGTCTGATTCGCGGCTATTTTGATCAGC
AGGGCATGCACATGCAGTTCAATGTTGTCGATCGCGCAACTCTGCTGGAT
GCTCAGGCGCACCCTGAGAAATACAGCGGTCTGATTGTTCGCGTGGCCGG
CTATTCTGCACTGTTCACCACTCTGTCTAAAAGCCTGCAGGACGATATAA
TAAAACGTACGGAACAAGCTGATAACCGTTAA

The RiDD-AE gene optimized for expression in E. coli is:

(SEQ ID NO: 4)
ATGAAGGAATATCTGAATACGAGCGGTCGCATCTTCGATATTCAACGTTA
TAGTATCCATGATGGGCCAGGCGTCCGCACAATCGTCTTTCTGAAAGGCT
GCGCACTGCGTTGTCGCTGGTGTTGTAATCCTGAAAGCCAAAGTTTTGAG
GTTGAAACCATGACTATTAACGGAAAACCAAAAGTGATGGGTAAAGATGT
AACCGTTGCCGAAGTTATGAAAACGGTTGAACGTGATATGCCATATTATC
TGCAATCTGGAGGTGGCATCACATTATCAGGTGGTGAATGCACATTACAG
CCGGAGTTTTCATTAGGACTGCTGCGTGCAGCAAAAGACTTAGGCATTTC
AACCGCTATCGAATCAATGGCGTATGCCAAGTACGAAGTAATAGAAACAC
TGCTGCCTTACCTGGATACGTATCTGATGGACATCAAGCACATGAACCCT
GAGAAACATAAAGAATATACCGGCCATGACAATCTGCGTATGCTGGAGAA
CGCTCTGCGCGTGGCGCATAGTGGTCAGACGGAATTAATTATTCGCGTTC
CTGTGATTCCGGGTTTTAACGCGACGGAACAGGAACTGTTAGACATTGCT
AAGTTTGCGGATACGTTACCGGGTGTCCGCCAGATCCATATCCTGCCATA
CCATAATTTCGGACAAGGCAAATACGAAGGCCTGAACCGTGATTACCCTA
TGGGAGACACGGAAAAGCCGAGCAACGAACAGATGAAGGCATTCCAGGAA
ATGATTCAAAAGAACACCAGTCTGCATTGTCAAATAGGAGGTTAA

The amino acid sequence for the RiDD described herein is:

(SEQ ID NO: 5)
MGNYDSTPIAKSDRIKRLVDHLYAKMPEIEAARAELITESFKATEGQPVV
MRKARAFEHILKNLPIIIRPEELIVGSTTIAPRGCQTYPEFSYEWLEAEF
ETVETRSADPFYISEETKKRLLAADAYWKGKTTSELATSYMAPETLRAMK
HNFFTPGNYFYNGVGHVTVQYETVLAIGLNGVKEKVRKEMENCHFGDADY
STKMCFLESILISCDAVITYANRYAKMAEEMAEKETDAARRQELLTIARV
CKNVPEFPAESFQEACQSFWFIQQVLQIESSGHSISPGRFDQYMYPYYEK
DLKEGSLTREYAQELIDCIWVKLNDLNKCRDAASAEGFAGYSLFQNLIVG
GQTVQGRDATNDLSFMCITASEHVFLPMPSLSIRVWHGSSKALLMRAAEL
TRTGIGLPAYYNDEVIIPALVHRGATMDEARNYNIIGCVEPQVPGKTDGW
HDAAFFNMCRPLEMVFSNGYDNGEIASIQTGNVESFQSFDEFMEAYRKQM
LYNIELMVNADNAIDYAHAKLAPLPFESCLVDDCIKRGMSAQEGGAIYNF
TGPQGFGIANVADSLYTIKKLVFEEKRITMGELKKALEMNYGKGLDATTA
GDIAMQVAKGLKDAGQEVGPDVIANTIRQVLEMELPEDVRKRYEEIHEMI
LELPKYGNDIDEVDELAREAAYFYTRPLETFKNPRGGMYQAGLYPVSANV
PLGAQTGATPDGRLAHTPVADGVGPTSGFDISGPTASCNSVAKLDHAIAS
NGTLFNMKMHPTAMAGEKGLESFISLIRGYFDQQGMHMQFNVVDRATLLD
AQAHPEKYSGLIVRVAGYSALFTTLSKSLQDDIIKRTEQADNR

The amino acid sequence for the RiDD-AE described herein is:

(SEQ ID NO: 6)
MKEYLNTSGRIFDIQRYSIHDGPGVRTIVFLKGCALRCRWCCNPESQSFE
VETMTINGKPKVMGKDVTVAEVMKTVERDMPYYLQSGGGITLSGGECTLQ
PEFSLGLLRAAKDLGISTAIESMAYAKYEVIETLLPYLDTYLMDIKHMNP
EKHKEYTGHDNLRMLENALRVAHSGQTELIIRVPVIPGFNATEQELLDIA
KFADTLPGVRQIHILPYHNFGQGKYEGLNRDYPMGDTEKPSNEQMKAFQE
MIQKNTSLHCQIGG

Each of the genes was placed in a high-copy bacterial replication vector for storage and maintenance of plasmid stocks. From these storage plasmids, PCR amplification of the codon-optimized genes was performed in order to introduce restriction sites compatible with the commercial yeast expression vector pESC-URA (catalog #217451, Agilent technologies). This vector contains cloning sites for the dual expression of two genes in yeast, specifically Saccharomyces cerevisiae, under strong constitutive promoters. Specifically, the pESC vectors are a series of epitope-tagging vectors designed for expression and functional analysis of genes in the yeast S. cerevisiae. These vectors contain the GAL1 and GAL10 yeast promoters in opposing orientation. In this case, we utilized the URA version in order to follow the successful recombination and rescue of the URA-phenotype. Yeast alcohol dehydrogenase is native to S. cerevisiae and is expressed under the anaerobic growth conditions described herein. Codon-optimized genes for the RiDD and the RiDD-AE were introduced into the commercially available pESC-URA vector to create the “pESC-Rose” vector. The empty vector is heretofore referred to as “pESC-empty”. Yeast cells harboring the pESC-Rose vector were then grown in minimal yeast drop-out URA-media on glucose under anaerobic conditions in the presence or absence of a racemic mixture of 1,2-propanediol. The pESC-empty vector was used as a negative control. These data are shown in FIG. 4.

The fermentation was performed in a 28 L fed-batch reactor under strictly anaerobic conditions. The reaction was initiated when 1 L of a healthy overnight culture (OD₆₀₀>1.5) was added to 9 L of media in the reactor. The feed rate was 0.5 liters per hour and the feedstock and initial media were as follows; basic YPD media that also contained 20 g/L glucose and 20 g/L 1,2-propanediol (racemic mixture). Ethanol and propanol production was monitored using the same GC equipped with a FID as described below.

Expression and Isolation of Catalytic Enzymes in E. coli

In order to produce sufficient enzyme for isolation and use in the cell-free reactor described herein, the genes for the Roseburi inulinivorans (RiDD) and the RiDD activating enzyme (RiDD-AE) were then amplified by standard PCR techniques using oligonucleotides that introduced a NheI and HindIII restriction site at the 5′- and 3′-end of the gene. This allows for sub-cloning into the commercially available pTRCHis™ vector and introduction of a 6× polyhistidine tail at the N-terminus with the following sequence “MSHHHHHGSG” (SEQ ID NO:7). Two expression vectors were created using this approach, one for the RiDD (pWNLRiDD) and one for the RiDD-AE (pWNLRiDDAE). Both expression vectors were standard E. coli expression containing T7 expression elements. The enzyme YqhD is native to E. coli and was cloned directly into the same vector after PCR amplification from crude extract using oligomers that introduced the same restriction sites at the 5′ and 3′ ends of the gene. The Ni—Fe hydrogenase was isolated from Ralstonia metallidurans or Ralstonia eutrophus (formally Alcaligenes eutrophus) by growing the native organism on a minimal media using fructose as the primary carbon source with glycerol as a secondary carbon source as previously described (REF). Expression of the RiDD and RiDD-AE in E. coli was accomplished by growing E. coli harboring the either the pWNLRiDD or pWNLRiDDAE plasmids in Terrific Broth (TB) at 37° C. with full aeration and the appropriate antibiotic until the cell density was 2.0 when measured at 600 nm. For cells carrying the pWNLRiDD plasmid, IPTG was added to a final concentration of 1.0 mM and the cells were allowed to incubate for an additional two hours before being harvested. For cells carrying the pWNLRiDDAE plasmid, ferrous ammonium citrate and IPTG were added to a final concentration of 0.5 mM and the cells were incubated an additional 12 hours at 18° C. with reduced aeration.

Isolation of Catalytic Enzymes and Use in a Cell-Free Reactor

The expression and purification of the Clostridium butyricum glycerol dehydratase (CbGD) and CbGD activating enzyme (CbGD-AE) was performed as previously described⁵. The RiDD and the RiDD-AE were purified using the same affinity column procedures reported for the butyricum enzymes⁵. Chemical reconstitution of [4Fe-4S] the catalytic [4Fe-4S] cluster of the RiDD-AE was performed as previously decribed⁸. This procedure was followed by anion exchange chromatography in order to remove adventitiously bound iron and sulfide. In short, the RiDD-AE was diluted to approximately 1 mg/mL in 20 mM TRIS pH 8.1, 50 mM KCl, and 10% glycerol. All solutions were made anaerobic by on a vacuum manifold with successive rounds of degassing and refilling with O₂-free argon. Sodium sulfide (0.2 mM), 2-mercapoethanol (5 mM), and ferrous ammonium sulfate (0.25 mM) were added to the solution and the entire mixture was gentle stirred overnight at 4° C. Any precipitate was removed by centrifugation and the supernatant was loaded onto a 5 mL Sepharose QFF column. The protein was then eluted from the column by applying a linear salt gradient (from 0 to 0.5 M KCl) in 20 mM TRIS pH 8.1. Elution typically occurred around 150 mM KCl. The RiDD-AE was concentrated and kept in liquid nitrogen. We noticed considerable enhancement of stability when the protein was stored in buffer containing either 10% glycerol or 1,3-propanediol.

The results from our “proof of concept” experiment are shown in FIG. 2. This experiment was performed in a 50 mL reactor with 20 mL liquid volume and the remaining headspace under 1 atm of oxygen-free hydrogen gas. Experimental conditions were as follows 20 mM Tris pH 7.9, 10% (v/v) 1,2-propanediol, 1% (v/v) glycerol 50 μM purified RiDD, 100 μM purified DhaT, 100 μM NAD+, and 100 μM purified H2ase. After a brief pre-incubation (20 min) period to insure strictly anaerobic conditions were achieved (less than 1 ppm O₂), purified RiDD-AE in the reduced (formally [4Fe-4S]′) form was added to a final concentration of 50 μM to initiate the reaction. At the time points indicated, 1 mL samples were extracted anaerobically and immediately treated with 250 μL of 1M formic acid. The acid treated samples were all centrifuged in a microfuge at 14,000 rpm for ten minutes in order to remove any precipitated material. A 1 μL volume of the acid-treated and centrifuged sample was injected into the GC and then separated using a temperature gradient that ramped from 60° C. to 120° C. over a 20 minute period. As the data in FIG. 2 show, the concentration of glycerol remained unchanged as the system continued to convert both enantiomers of 1,2-propanediol into propanol (˜14 g/L after 6 hours). Despite the high degree of sequence identity to previously characterized B₁₂-dependent dehydratases found in existing art and the current patent literature, the “wild-type” form of the RiDD is unique in that it is specific for 1,2-propanediol and will not touch glycerol. This is a very important distinction since glycerol is a very important metabolic chemical associated with anaerobic metabolism in a number of microorganisms including S. cerevisiae and E. coli.

### Enzyme Assay for the CbGD and the RiDD.

A coupled enzyme assay for the CbGD and the RiDD was performed as previously described⁵ with minor modifications. Where indicated, varying amounts of the substrate (specifically, glycerol, R-1,2-propanediol, or S-1,2-propanediol) were added from pure stock solutions purchased through Sigma Chemical Company™ in some cases the stocks were diluted with ddH₂O in order to allow for accurate measurement of the substrate at the lower concentrations. In addition, when glycerol was used as the substrate for the CbGD, the enzyme YqhD was included in the assay in place of yeast alcohol dehydrogenase (YADH) and NADPH was replaced with NADPH. YqhD is an aldehyde reductase capable of the NADPH-dependent reduction of several aldehydes, including 3-hydroxypropionaldehyde and was overexpressed and purified from E. coli as previously described⁹.

### Direct Detection of Dehydratase Activity.

The direct detection of propionaldehyde, 1,2-propanediol, and acetone was performed using a gas chromatograph (GC) equipped with a 30 m×0.329 mm Carbowax 20M column. In these assays, the coupled assay reported by Obrien et al. was simply scaled up to 40 mL and YADH as well as NADH was omitted from the assay. In all cases, the amount of dehydratase in the assay was approximately 1 μM. In addition, the substrate (either R- or S-1,2-propandiol) was 5% (v/v) at the beginning of the assay and all assays were initiated by the addition of the purified activating enzyme (either CbGD-AE or RiDD-AE). At the time points indicated, 1 mL samples were extracted anaerobically and immediately treated with 250 μL, of 1M formic acid. The acid treated samples were all centrifuged in a microfuge at 14,000 rpm for ten minutes in order to remove any precipitated material. A 1 μL, volume of the acid-treated and centrifuged sample was injected into the GC and then separated using a temperature gradient that ramped from 60° C. to 120° C. over a 20 minute period. The GC was equipped with a flame ionization detector and a series of standards for 1,2-propanediol, acetone, or propionaldehyde was used to calibrate the integrated peak areas.

### Crystallization, Data Collection, and Structure Determination

Initial crystallization conditions for the RiDD and YqhD enzymes were identified in sitting drop experiments and then optimized in a hanging-drop tray. The final conditions for diffraction quality crystals of the RiDD were 0.125 M sodium acetate, 42% PEG 400 (w/v), and 0.025 M HEPES pH 7.5. Crystallization was performed in hanging drop experiments with 800 μL of precipitating solution in the well. Protein (40 mg/mL) and precipitation solution were mixed (2 μL each) to initiate the reaction and then the tray was placed at 4° C. for 12 hours before being moved to an incubator at 18° C. Crystals of the RiDD took approximately one week to form and no further cryo-protection was required for freezing. Crystals of purified YqhD were obtained using a precipitating solution consisting of 0.3 M NaCl, 25% (w/v) PEG 3000, and 0.1 M TRIS pH 7.2. Crystallization of YqhD was also performed in hanging drop experiments with 800 μL of precipitating solution in the well. Purified YqhD (25 mg/mL) and precipitation solution were mixed (2 μL each) to initiate the reaction and then the tray was placed directly in an incubator at 18° C. Crystals typically formed overnight or within the first 48 hours.

TABLE 1
Data collection and refinement statistics.

| Enzyme | RiDD | YqhD |
|---|---|---|
| Space Group | C222₁ | C2 |
| Wavelength | 0.98 | 0.98 |
| Resolution Range (\acute{Å}) | 50.0-2.1 | 50.0-1.7 |
| Outer Shell | 2.18-2.1 | 1.76-1.7 |
| Unique Observations | 166,139 | 44,687 |
| Completeness (%) | 98.9 (90.3)^{a} | 96.3 (82.5) |
| R_{sym} (%)^{b} | 0.06 (0.29) | 0.04 (0.19) |
| Redundancy | 6.9 (3.4) | 3.8 (3.3) |
| I/σ | 22.2 (2.8) | 35.6 (3.6) |
| Enzyme | RiDD | YqhD |
| Unit Cell (a, b, c) in \acute{Å} | 138, 183, 228 | 102, 68, 66 |
| Protein Atoms | 13,127 | 2,959 |
| Solvent Atoms | 1,153 | 326 |
| Resolution Limits (\acute{Å}) | 50.0-2.1 | 50.0-1.7 |
| R_{cryst} (%) | 15.2 | 18.1 |
| R_{free} (%) | 19.0 | 21.8 |
| rmsd bonds (\acute{Å}) | 0.007 | 0.007 |
| rmsd angles (°) | 1.01 | 1.10 |
| average B factor (\acute{Å}²) | 30.2 | 29.4 |

^{a}Numbers in parentheses denote values for the outermost resolution shell.
^{b}R_{sym} = Σₕₖₗ[Σ_{I}(|I_{hkl,I} − <Iₕₖₗ>|)]/Σ_{hkl,I}<Iₕₖₗ>, where Iₕₖₗ is the intensity of an individual measurement of the reflection with indices hkl and <Iₕₖₗ> is the mean intensity of that reflection.

Data was collected at the Advance Photon Source through SER-CAT and beam line 22BM at 0.98 Å. The program PHENIX¹⁰ was used to solve the initial phase problem with a poly-alanine model of the CbGD (PDB ID 1R9D) and YqhD (PDB ID 1OJ7) serving as the search models where appropriate. Iterative rounds of refinement using COOT¹¹ and PHENIX were performed with experimental phase restraints and a 5.0% R_{free} test set¹² that was generated by PHENIX and used throughout all stages of refinement.

Results

Substrate and Enantiomer Specificity for the CbGD and RiDD.

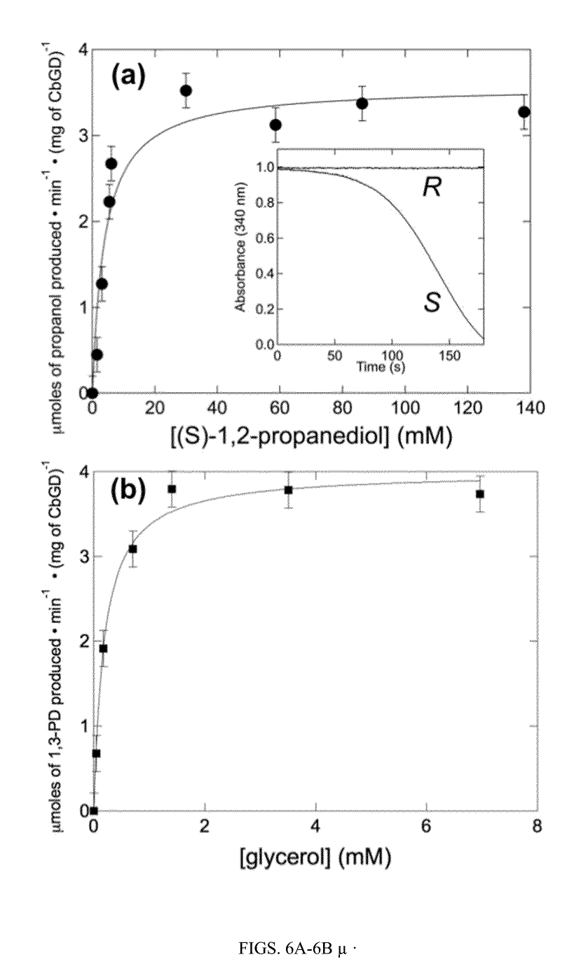

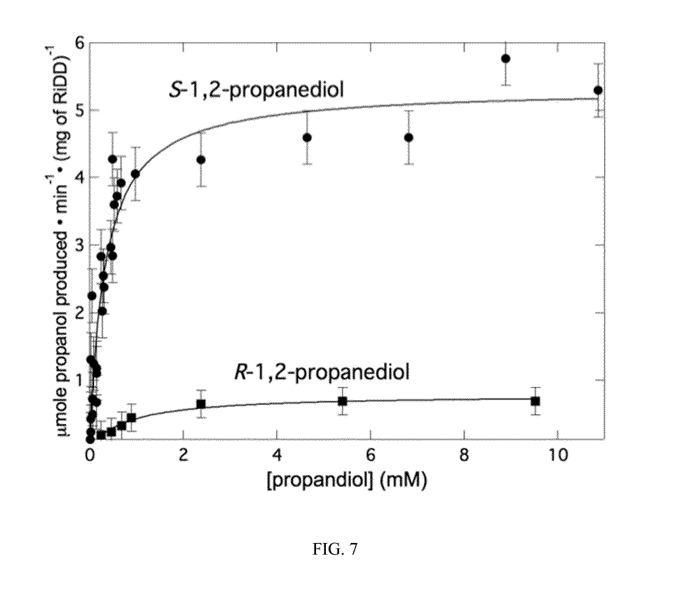

In the preliminary characterization of the CbGD, a racemic mixture of 1,2-propandiol was used in order to assay the enzyme⁵. In addition, the same racemic mixture of 1,2-propanediol was used to treat protein crystals of the CbGD for cryo-crystallography. In fact, one advantage to working with this enzyme is that both substrates (glycerol and 1,2-propanediol) are also good cryo-protectants. In the original structure of the 1,2-propandiol-bound form of the CbGD we had modeled in the “R” enantiomer. We have since re-analyzed the data and see a minor improvement in the R-factors and difference maps for the model when S-1,2-propanediol is modeled in the active site. We have already deposited the corrected structure in the protein data bank (4MTJ), but the question as to whether or not the CbGD actually prefers one enantiomer and not the other as a substrate has not been determined. In order to address this, we assayed the CbGD using either R-1,2-propanediol or S-1,2-propanediol, these results are shown in FIG. 6 (Panel A), along with the results using the physiological substrate glycerol (FIG. 6, Panel B). These results clearly show that the CbGD can utilize glycerol and 5-1,2-propanediol as a substrate. No activity was observed when R-1,2-propanediol was used instead of S-1,2-propanediol. As one would expect, the Kₘ for the physiological substrate, glycerol, is lower than that of S-1,2-propanediol (approximately 0.2 mM for glycerol compared with 0.8 mM for 5-1,2-propanediol). In addition to our previous work, these findings further expand our catalytic knowledge of the CbGD and are consistent several patents that have been issued for this enzyme (WO 2001004324; WO 2013085321). In contrast to the activity observed for the CbGD, kinetic analysis of the RiDD revealed that this dehydratase will not utilize glycerol as a substrate but, somewhat surprisingly, will accept both enantiomers of 1,2-propanediol as a substrate (FIG. 7). These findings are consistent the predictions made by Scott et al. and the metabolic activity that has been reported for Roseburia inulinivorans. Specifically, Scott et al. detected the anaerobic production of propanol by this organism. Presumably the final metabolic step would be the NADH- or NADPH-dependent reduction of propionaldehyde by an aldehyde reductase, but more specifically, no 1,3-propanediol (1,3-PD) production was detected for R. inulinovaroans¹³ even under media conditions that favor production of 1,3-PD. In addition to confirming that 1,2-propanediol is a viable substrate for the RiDD, our data also show that S-1,2-propanediol is the preferred enantiomer for the RiDD. Fitting the data to Michaelis-Menten equation indicates a similar Kₘ of approximately 0.5 mM for S-1,2-propanediol and 0.8 mM for R-1,2-propanediol. However, as can be seen, the maximum specific activity is considerably higher for the “S” enantiomer. An explanation for the substrate and enantiomer specificity observed for the CbGD and the RiDD is not immediately apparent. This is in large part due to the 54% identity observed when the primary structure of the CbGD and RiDD are aligned.

Acetone Production by the RiDD.

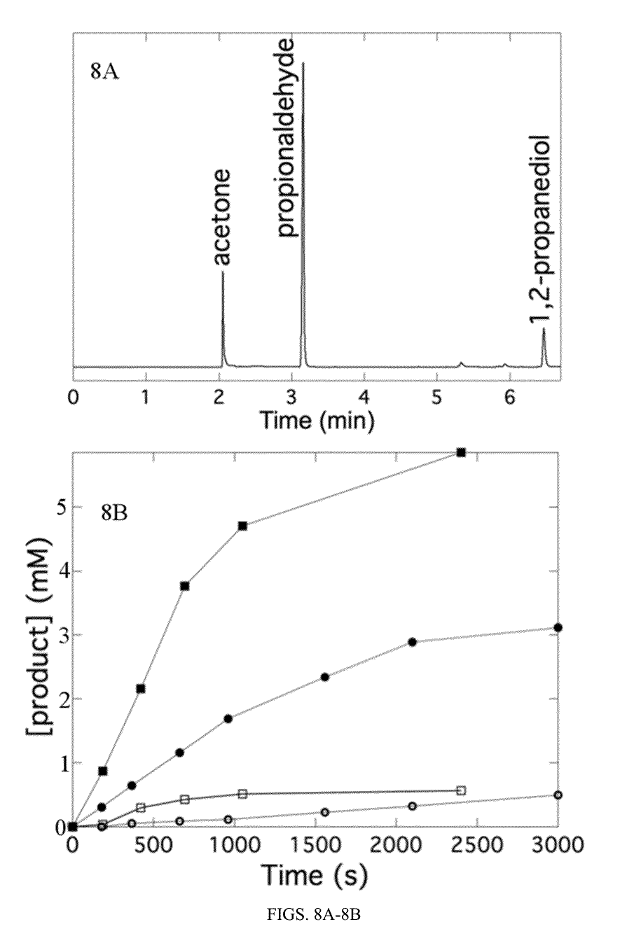

An inherent weakness of the coupled assay is the fact that, for the majority of substrate concentrations investigated, the NADH in the assay will be consumed long before the primary substrate (either glycerol or 1,2-propanediol) is exhausted. In this case, there are alternative approaches to directly monitoring the activity of the dehydratase. Of significance to the activity being examined in this work is a recent patent that was focused on another B₁₂-independent dehydratase. The patent was issued to LANZATECH New Zealand Limited (International Publication Number WO2014/036152). The authors claim to have identified a new “stereospecific dehydratase” that will produce either acetone or propionaldehyde depending on whether or not the substrate is R-1,2-propanediol or S-1,2-propanediol, respectively. According to Mueller and Koepke, the enzyme was found in Clostridium autoethanogenum and was a novel diol dehydratase (CaDD), similar to the RiDD we characterize in this work. Given the stereo-selective dehydration of the enantiomers of 1,2-propanediol we observe for the CbGD and the activity of the RiDD described in the previous section, we were interested in determining whether or not any acetone production was observed for the CdGD or the RiDD. In order to determine the relative levels of propionaldehyde or potentially any acetone production, we used a gas chromatograph (GC) equipped with a flame ionization detector (FID) to directly measure these compounds. Similar to what others have previously reported¹⁴ the application of a GC-FID and the appropriate column gives very reliable separation of these small aldehydes, ketones, or alcohols (FIG. 8, Panel A). No detectable acetone production was observed for the CbGD, however, when the RiDD was assayed with either enantiomer 1,2-propanediol, we did observe some level of acetone production (FIG. 8, Panel B). This is in contrast to the patent reported for what is most likely a homologous enzyme in C. autoethanogenum. What isn't known is the relative positioning of conserved residues in the active site for all three enzymes. This may explain the different substrate and enantiomer selectivity. This is investigated further and described in the following sections. Both the “cell free” system and the expression of the dehydratase system in yeast described herein provide a mechanism to remove the aldehyde product quickly, preventing any significant acetone accumulation.

The Active Site Structure of the RiDD.

In addition to the similarities in secondary and tertiary structure revealed above, the RiDD has 48% sequence identity when compared to the CbGD. FIG. 9 shows an overlay comparing the active site structures observed in the crystallographic model for the CdGD with glycerol bound and the RiDD model determined in this work. Two important observations are immediately apparent when considering the new structural information. First, very low molecular weight PEGs provided the precipitant for crystallization of the RiDD and we serendipitously appear to have trapped ethane diol in the active site of the RiDD (FIG. 9). Consistent with existing art, we predict that the hydroxyl groups of this diol are bound in the active site in a manner identical to that of the hydroxyl groups found on 1,2-propanediol. Secondly, when considering the amino acids that can interact with the substrates glycerol and 1,2-propanediol, the data in FIG. 9 show that all but two of the amino acids found in the active site of the CbGD are conserved in the active site of the RiDD. For the CbGD the residues of interest are Y339 and S642. The equivalent residues are F344 and V696 in the RiDD, respectively. An important discovery is seen in the crystallographic model for the RiDD reported here when this data is compared to the CbGD model (FIG. 9, Panel B). Specifically, it is clear that the V696 side chain of the RiDD would occlude the space required for a third hydroxyl group on the substrate, hence, explaining why glycerol is not a substrate for this dehydratase. In this case, glycerol may simply not “fit” into the active site of the RiDD. Moreover, the presence of an additional hydrogen bond between the side chain of Y339 and S642 found in the active site of the CbGD will also provide more “rigidity” to the active site. In contrast, the absence of that hydrogen bond will allow for some rotational flexibility and the accommodation of both enantiomers of 1,2-propanediol.

### REFERENCES

1. Selmer, T., Pierik, A. J. & Heider, J. New glycyl radical enzymes catalysing key metabolic steps in anaerobic bacteria. Biol Chem 386, 981-8 (2005).
2. Krieger, C. J., Roseboom, W., Albracht, S. P. & Spormann, A. M. A stable organic free radical in anaerobic benzylsuccinate synthase of Azoarcus sp. strain T. J Biol Chem 276, 12924-7 (2001).
3. Selmer, T. & Andrei, P. I. p-Hydroxyphenylacetate decarboxylase from Clostridium difficile. A novel glycyl radical enzyme catalysing the formation of p-cresol. Eur J Biochem 268, 1363-72 (2001).
4. Sun, X. et al. Generation of the glycyl radical of the anaerobic Escherichia coli ribonucleotide reductase requires a specific activating enzyme. J Biol Chem 270, 2443-6 (1995).
5. O'Brien, J. R. et al. Insight into the mechanism of the B12-independent glycerol dehydratase from Clostridium butyricum: preliminary biochemical and structural characterization. Biochemistry 43, 4635-45 (2004).
6. Craciun, S. & Balskus, E. P. Microbial conversion of choline to trimethylamine requires a glycyl radical enzyme. Proc Natl Acad Sci USA 109, 21307-12 (2012).
7. Eklund, H. & Fontecave, M. Glycyl radical enzymes: a conservative structural basis for radicals. Structure 7, R257-62 (1999).
8. Kulzer, R., Pils, T., Kappl, R., Huttermann, J. & Knappe, J. Reconstitution and characterization of the polynuclear iron-sulfur cluster in pyruvate formate-lyase-activating enzyme. Molecular properties of the holoenzyme form. J Biol Chem 273, 4897-903 (1998).
9. Sulzenbacher, G. et al. Crystal structure of E. coli alcohol dehydrogenase YqhD: evidence of a covalently modified NADP coenzyme. J Mol Biol 342, 489-502 (2004).
10. Adams, P. D. et al. PHENIX: a comprehensive Python-based system for macromolecular structure solution. Acta Crystallogr D Biol Crystallogr 66, 213-21 (2010).
11. Emsley, P. & Cowtan, K. Coot: model-building tools for molecular graphics. Acta Crystallogr D Biol Crystallogr 60, 2126-32 (2004).
12. Brunger, A. T. Free R value: a novel statistical quantity for assessing the accuracy of crystal structures. Nature 355, 472-5 (1992).
13. Scott, K. P., Martin, J. C., Campbell, G., Mayer, C. D. & Flint, H. J. Whole-genome transcription profiling reveals genes up-regulated by growth on fucose in the human gut bacterium “Roseburia inulinivorans”. J Bacteriol 188, 4340-9 (2006).
14. Pontes, H. et al. GC determination of acetone, acetaldehyde, ethanol, and methanol in biological matrices and cell culture. J Chromatogr Sci 47, 272-8 (2009).

We claim:

1. A non-naturally occurring microorganism comprising an exogenous diol dehydratase enzyme.

2. The non-naturally occurring microorganism of claim 1, wherein the enzyme is at least 80% identical in protein sequence to Roseburia inulinivorans diol dehydratase (RiDD).

3. The non-naturally occurring microorganism of claim 1, wherein the enzyme is Roseburia inulinivorans diol dehydratase (RiDD).

4. The non-naturally occurring microorganism of claim 1 that does not convert glycerol to 3-hydroxypropionaldehyde.

5. The non-naturally occurring microorganism of claim 1 that does not convert propanal to propanol.

6. The non-naturally occurring microorganism of claim 1, comprising an exogenous nucleic acid that encodes for the enzyme.

7. The non-naturally occurring microorganism of claim 1, wherein the microorganism is in a substantially anaerobic culture medium.

8. The non-naturally occurring microorganism of claim 1, further comprising an exogenous diol dehydratase activating enzyme in an amount sufficient to activate the diol dehydratase enzyme.

9. The non-naturally occurring microorganism of claim 8, further comprising an exogenous nucleic acid that encodes for a diol dehydratase activating enzyme.

10. The non-naturally occurring microorganism of claim 9, wherein the diol dehydratase activating enzyme is at least 80% identical in protein sequence to Roseburia inulinivorans diol dehydratase activating enzyme (RiDD-AE).

11. The non-naturally occurring microorganism of claim 1, further comprising an alcohol dehydrogenase enzyme.

12. The non-naturally occurring microorganism of claim 1 that converts both enantiomers of 1,2-propanediol to propanal.

13. A cell-free enzyme mixture comprising:
an isolated diol dehydratase enzyme capable of converting both enantiomers of 1,2-propanediol to propanal; and
an alcohol dehydrogenase.

14. The cell-free enzyme mixture of claim 13, wherein the diol dehydratase enzyme is at least 80% identical in protein sequence to Roseburia inulinivorans diol dehydratase (RiDD).

15. The cell-free enzyme mixture of claim 13, wherein the diol dehydratase is Roseburia inulinivorans diol dehydratase (RiDD).

16. The cell-free enzyme mixture of claim 13, further comprising a diol dehydratase activating enzyme that activates the diol dehydratase enzyme to convert both enantiomers of 1,2-propanediol to propanal.

17. The cell-free enzyme mixture of claim 16, wherein the diol dehydratase activating enzyme is at least 80% identical in protein sequence to Roseburia inulinivorans diol dehydratase activating enzyme (RiDD-AE).

18. A method of making a non-naturally occurring microorganism according to claim 1, the method comprising:
expressing a nucleic acid in the microorganism that encodes for at least a diol dehydratase; and
culturing the microorganism for a suitable period of time and in a suitable culture medium to produce the microorganism.

19. The method of claim 18, wherein the diol dehydratase converts both enantiomers of 1,2-propanediol to propanal.

20. A method of converting 1-2-propanediol to propanol, comprising culturing a microorganism according to claim 1 under conditions and for a period of time sufficient to convert the 1,2-propanediol to propanol.

21. The method of claim 20, wherein the conditions comprise a substantially anaerobic culture medium.

22. The method of claim 20, wherein the 1,2-propanediol comprises at least (S)-1,2-propanediol.

23. The method of claim 20, wherein the 1,2-propanediol comprises a racemic mixture of both enantiomers of the 1,2-propanediol.
